(19) United States
(12) Patent Application Publication
(10) Pub. No.: US 2001/0006939 A1
(43) Pub. Date: Jul. 5, 2001

(54) SECRETORY LEUKOCYTE PROTEASE INHIBITOR DRY POWDER PHARMACEUTICAL COMPOSITIONS

(76) Inventors: RALPH W. NIVEN, REDWOOD CITY, CA (US); CLIFFORD D. WRIGHT, BOULDER, CO (US); BYEONG S. CHANG, THOUSAND OAKS, CA (US)

Correspondence Address: AMGEN INCORPORATED, MAIL STOP 27-4-A, ONE AMGEN CENTER DRIVE, THOUSAND OAKS, CA, 91320-1799, US

(21) Appl. No.: 08/943,759

(22) Filed: Oct. 3, 1997

(51) Int. Cl. A01N 37/18; C12P 21/06; A01N 43/04

(52) U.S. Cl. 514/2; 514/44; 435/69.1

(57) ABSTRACT

The present invention relates to the pulmonary administration of a therapeutic protein by means of powdered pharmaceutical compositions suitable for inhalation therapy. In particular the invention relates to dry powder formulations of secretory leukocyte protease inhibitor (SLPI) for pulmonary delivery. Exemplary pharmaceutical compositions contain SLPI and a pharmaceutically acceptable carrier in the form of a dry powder which is typically less than about 10% by weight water. About 50% to 95% by mass of the powder comprises particles or agglomerates of particles having a diameter within the range of from about 1.0 microns to about 8 microns, with a mass median diameter ranging from about 3.0 microns to about 6 microns.

### FIELD OF THE INVENTION

The present invention relates to the pulmonary administration of a therapeutic protein by means of powdered pharmaceutical compositions suitable for inhalation therapy. In particular the invention relates to dry powder formulations of secretory leukocyte protease inhibitor for pulmonary delivery.

### BACKGROUND OF THE INVENTION

Endogenous proteolytic enzymes serve to degrade invading organisms, antigen-antibody complexes and certain tissue proteins which are no longer necessary or useful to the organism. In a normally functioning organism, proteolytic enzymes are produced in a limited quantity and are regulated in part through the synthesis of protease inhibitors.

A large number of naturally occurring protease inhibitors serve to control the endogenous proteases by limiting their reactions locally and temporally. In addition, the protease inhibitors may inhibit proteases introduced into the body by infective agents. Tissues that are particularly prone to proteolytic attack and infection, e.g., those of the respiratory tract, are rich in protease inhibitors.

A disturbance of the protease/protease inhibitor balance can lead to protease-mediated tissue destruction, including emphysema, asthma, arthritis, glomerulonephritis, periodontitis, muscular dystrophy, tumor invasion and various other pathological conditions. In certain situations, e.g., severe pathological processes such as sepsis or acute leukemia, the amount of free proteolytic enzymes present increases due to the release of enzyme from the secretory cells. A diminished regulating inhibitor capacity of the organism may also cause alterations in the protease/protease inhibitor balance.

In organisms where such aberrant conditions are present, serious damage to the organism can occur unless measures can be taken to control the proteolytic enzymes. Therefore, protease inhibitors have been sought which are capable of being administered to an organism to control the proteolytic enzymes.

Where the concern is a disease state of the lungs, the protease inhibitor may be directly delivered to the diseased tissue by aerosolization of a drug solution (such as by a nebulizer) and subsequent inhalation of the aerosol droplets containing the drug. However, even where one directs the drug solution to the lungs initially, there are substantial uncertainties about efficacy in treating the lungs. For example, the half-life of the drug in the lungs may be relatively short due to absorption into the vascular system. In addition, those drugs which are sensitive to enzymatic degradation or other processing, will be subject to modification and loss of efficacy. There is also the problem of the effect of aerosolization on the drug, where the drug may be degraded by the nebulizing action of the nebulizer or inactivated by oxidation. There is also the uncertainty concerning the distribution of the drug in the lungs, as well as the ability to maintain an effective dosage for an extended period, without detrimental effect to the lungs or other organs of the host.

There has been some prior success in the pulmonary administration of pharmaceutical compositions containing low molecular weight drugs, most notably in the area of beta-androgenic antagonists to treat asthma. Other low molecular weight, non-proteinaceous compounds, including corticosteroids and cromolyn sodium, have been administered systemically via pulmonary absorption. Not all low molecular weight drugs, however, can be efficaciously administered through the lung. For instance, pulmonary administration of aminoglycoside antibiotics, anti-viral drugs and anti-cancer drugs for systemic action has met with mixed success. In some cases, the drug was found to be irritating and bronchoconstrictive. Thus, even with low molecular weight substances, it is not at all predictable that the pulmonary delivery of such compounds will be an effective means of administration. See generally Peptide and Protein Drug Delivery, ed. V. Lee, Marcel Dekker, N.Y., 1990, pp. 1-11.

Pulmonary delivery of higher molecular weight pharmaceuticals, such as proteins, is not unknown, although only a few examples have been quantitatively substantiated. Leuprolide acetate is a nonapeptide with luteinizing hormone releasing hormone (LHRH) agonist activity having low oral availability. Studies with animals indicate that inhalation of an aerosol formulation of leuprolide acetate results in meaningful levels in the blood. (See Adjel et al., Pharmaceutical Research, Vol. 7, No. 6, pp. 565-569 (1990); Green, J. D., 1994. Pharmacotoxicological expert report: Pulmozyme™. rhDNase. Genentech, Inc. Human & Experimental Toxicology vol. 13; suppl. 1.)

The feasibility of delivering human plasma alpha-1-antitrypsin to the pulmonary system using aerosol administration, with some of the drug gaining access to the systemic circulation, is reported by Hubbard et al., Annals of Internal Medicine, Vol. 111, No. 3, pp. 206-212 (1989). The aerosol administration of liquid formulations of alpha-1-antitrypsin by means of a nebulizer is further described in U.S. Pat. No. 5,618,786. However, vasoactive intestinal peptide, a small polypeptide with a molecular weight of 3,450 daltons (D) which causes bronchodilation when given intravenously in animals, including humans, lacks efficacy when administered by inhalation. See Barrowcliffe et al., Thorax, vol. 41(2):88-93, 1986.

As demonstrated by these examples of protein delivery via the pulmonary route, it is not predictable whether a given protein delivered in such a manner will have a therapeutic effect. Nor is it predictable that a protein may be formulated for delivery in a dry powder form yet retain its biological activity. Various factors intrinsic to the protein itself, the pharmaceutical composition, the delivery device, and particularly the lung, or a combination of these factors, can influence the success of pulmonary administration.

One protease inhibitor of particular interest for the treatment of pulmonary diseases is secretory leukocyte protease inhibitor (SLPI). SLPI is a selective inhibitor of serine proteases, including tryptase, elastase, chymase, and cathepsin G. Evaluations of SLPI activity indicate that the protein may be used in the treatment of lung diseases characterized by excess levels of proteases as well as by leukocyte- or mast cell-mediated disorders. Descriptions of purification, recombinant production, synthesis, and the identification of SLPI truncation, addition and substitution analogs are described in U.S. Pat. No. 4,760,130 (Thompson et al.), U.S. Pat. No. 4,845,076 (Heinzel et al.), U.S. Pat. No. 5,290,762 (Lezdey et al.) and EP 346 500 (Teijin). U.S. Pat. No. 5,618,786 (Roosdorp et al.) and WO 96/08275 (Bayer) disclose the pulmonary delivery of liquid formulations of serine protease inhibitors. Such formulations involve the use of a nebulizer which mechanically creates a mist of fine droplets from a solution or suspension of a drug, wherein the mist is inhaled through the mouth and/or nose by the patient. Vogelmeier et al. (Journal of Applied Physiology, 69(5):1843-1848, 1990) and Stolk et al. (Thorax, 50(6):645-650, 1995) describe the use of liquid SLPI formulations that may be delivered by means of a nebulizer. The inventors, however, are unaware of any prior reports of the preparation and inhalation delivery of an effective dry powder pharmaceutical composition containing SLPI.

In addition to delivery by liquid nebulizers, pulmonary drug delivery can be achieved with aerosol-based metered-dose inhalers (MDI's). These devices typically involve a pressurized canister from which the drug/propellant formulation may be released. Conventional propellants include fluorohydrocarbons. Once released, the propellant evaporates and particles of the drug are inhaled by the patient.

The liquid preparations which require a nebulizer or atomizer are not readily transportable or easy to use. The fluorohydrocarbon aerosol preparations are easier to handle, and have been widely used, but they typically rely on the use of chlorofluorocarbons (CFC's), which may have adverse environmental effects. Hydrocarbon propellants have replaced the fluorocarbons for some aerosol formulations, but they may have limited use due to flammability. Under such circumstances, dry powder dispersion devices, which do not rely on CFC aerosol technology, are promising for delivering drugs that may be formulated as dry powders.

Certain proteins and polypeptides may be stably stored as lyophilized powders by themselves or in combination with suitable powder carriers. The ability to deliver dry powder proteins and polypeptides as a therapeutic entity, however, is unpredictable and problematic in certain respects. The dosage of many protein and polypeptide drugs is often critical so it is necessary that any dry powder delivery system be able to accurately, precisely, and reliably deliver the intended amount of drug. Moreover, many proteins and polypeptides are quite expensive, typically being many times more costly than conventional drugs on a per-dose basis. Thus, the ability to deliver the dry powders with a minimal loss of drug is critical. It is also important that the powder be readily dispersible prior to inhalation by the patient in order to assure adequate distribution.

Another requirement for protein powder delivery is efficiency. It is important that the concentration of drug in the bolus of gas be relatively high to reduce the number of breaths required to achieve a total dosage. The ability to achieve both adequate dispersion and small dispersed volumes is a significant technical challenge that requires in part that each unit dosage of the powdered composition be readily and reliably dispersible.

Yet another aspect of efficient delivery involves particle size. The particle size for efficient delivery should be within the micrometer range and should be between 1 and 8 μm with a mass median diameter of between about 3 and 6 μm (J. Pharm. Sci., 1986;75:433). Devices for controlling the particle size of an aerosol upon delivery are known. For example, U.S. Pat. No. 5,522,385 describes a mechanical device for the control of particle size upon delivery rather than upon formulation. A device is provided which creates aerosolized particles by moving a drug through a nozzle in the form of a porous membrane with sufficient energy added to evaporate a carrier and thereby reduce particle size. U.S. Pat. No. 4,790,305 describes the control of particle size of a metered dose of aerosol by filling a first chamber with medication and a second chamber with air such that all of the air is inhaled prior to inhaling the medication, and using flow control orifices to control the flow rate. U.S. Pat. No. 4,926,852 refers to metering a dose of medication into a flow-through chamber that has orifices to limit the flow rate to control particle size. U.S. Pat. No. 4,677,975 refers to a nebulizer device that uses baffles to remove from the aerosol droplets those particles above a selected size. U.S. Pat. No. 3,658,059 refers to a baffle that changes the size of an aperture in the passage of the suspension being inhaled to select the quantity and size of suspended particles delivered. A problem with these devices is that they process the aerosol after it is generated, and thus, are inefficient.

### SUMMARY OF THE INVENTION

The present invention is based upon the unexpected discovery that secretory leukocyte protease inhibitor (SLPI) may be delivered in a therapeutically efficacious manner by direct administration of the dry powder protein to the lungs of a patient (hereinafter “pulmonary administration”). In addition, SLPI delivered to the lungs in this manner may be specifically formulated for enhanced intratracheobronchial delivery (airway delivery to the trachea, bronchi and bronchioles). This new means of SLPI administration enables the rapid delivery of a specified medicament dosage to a patient without the necessity for injection. In addition, pulmonary administration more readily lends itself to self-administration by the patient. Moreover, the pharmaceutical compositions of the present invention provide readily dispersible dry powder particles which are suitable for inhalation and which accurately, precisely, and reliably deliver the intended amount of SLPI protein to the most advantageous site for therapeutic effect. In addition, the present compositions provide for the efficient delivery of SLPI protein with minimal loss per unit dosage form. Examples of mechanical devices useful in accordance with the methods of the invention include metered dose inhalers and powder inhalers.

Mast cell tryptase and neutrophil elastase levels are elevated in asthmatic airways and contribute to bronchoconstriction and airway hyperresponsiveness. The serine proteases also promote pathologic changes to asthmatic airways including mucous hypersecretion (cathepsin G, elastase), epithelial cell desquamation (cathepsin G, elastase), edema (tryptase) and smooth muscle hyperplasis (tryptase). The spray-dried pharmaceutical compositions of the present invention provide a serine protease inhibitor, SLPI, which inhibits the activity of these proteases.

In one embodiment, the present invention provides a pharmaceutical composition, comprising SLPI and a pharmaceutically acceptable carrier, wherein the composition is a dry powder of less than about 10% by weight water, and wherein 50% to 95% by mass of the powder comprises particles or agglomerates of particles having a diameter within the range of from about 1.0 microns to about 8 microns with a mass median diameter ranging from about 3.0 microns to about 6 microns. In a preferred composition, the particles are at least 50% dispersible in a current of a gas. In a more preferred composition, the particles or agglomerates of particles have a mass median diameter ranging from about 4.5 microns to about 5.5 microns.

Typically, SLPI comprises from about 50 to about 95 percent by weight of the composition, and the pharmaceutically acceptable carrier comprises from about 5 to about 50 percent by weight of the composition. The pharmaceutically acceptable carrier may be a carbohydrate, amino acid or polypeptide. Exemplary pharmaceutically acceptable carriers include mannitol, sucrose, and trehalose. The pharmaceutical composition may include further pharmaceutical excipients, such as a dispersing agent and/or an absorption enhancer. In a preferred embodiment the pharmaceutical composition is produced by providing a mixture of SLPI and optionally a pharmaceutically acceptable carrier in a solvent, and spray drying the mixture to form a dry powder.

The novel formulations of the present invention result in pharmaceutical compositions which increase the amount of SLPI protein that can be delivered (i.e., higher drug load per particle) and increase the delivery to the desired target area, i.e., large airways (bronchi, bronchioles), by means of the uniform particle size. This results in compositions which may provide increased efficacy and duration of action while decreasing the required dose to be delivered as well as the dosage schedule. In addition, the novel compositions have enhanced storage stability and are more conveniently used.

SLPI protein may be obtained from natural sources, or more preferably it is produced either by protein synthesis or recombinant technology techniques. In a particularly preferred embodiment, recombinantly produced SLPI protein is used in the pharmaceutical compositions.

In the preferred embodiments, the present invention features compositions and methods for the inhibition of leukocyte or mast cell serine proteases including elastase, tryptase, and cathepsin G. The compositions would be beneficial in the treatment of lung diseases characterized by excess levels of proteases as well as by leukocyte- and mast cell-mediated disorders. The compositions would be particularly beneficial in the treatment of inflammatory airway diseases such asthma, chronic bronchitis, chronic obstructive pulmonary disease, emphysema, as well as other forms of bronchoconstriction, of acute respiratory failure, or of reversible pulmonary vasoconstriction (i.e., acute pulmonary vasoconstriction or chronic pulmonary vasoconstriction which has a reversible component). Therapies could also include the treatment of other airway disorders such as infectious disease indications, oncology, pulmonary hypertension, etc. The compositions may be used in the treatment of pulmonary diseases characterized by increased pulmonary mucous production/secretion, decreased mucous velocity in the airways, increased airway hyperresponsiveness to antigen/stimulus and/or pathological changes in airway cells/tissue.

### BRIEF DESCRIPTION OF THE DRAWINGS

FIG. 1. Depicts the effect of secretory leukocyte protease inhibitor (SLPI) on antigen-induced airway hyperreactivity in guinea pigs. Hyperreactivity is determined as the shift of the dose-dependent bronchoconstriction (assessed as Pause_{enhanced}) to histamine evaluated 6 hours after antigen challenge (mean±SE, n=10) (#p<0.05 antigen-stimulated response vs. baseline values). FIG. 1a. Single dose intratracheal instillation of SLPI one hour before antigen challenge inhibits the development of hyperresponsiveness (mean±SE, n=4-6) (+p<0.1 effect of SLPI vs. antigen-stimulated response) (*p<0.05 effect of SLPI vs. antigen-stimulated response). FIG. 1b. Intratracheal instillation of SLPI daily for 2 days and one hour before antigen challenge (predosing regimen) increases the potency for inhibition of the development of hyperresponsiveness (mean±SE, n=6) (*p<0.05 effect of SLPI vs. antigen-stimulated response).

FIG. 2. Depicts the prolonged activity of SLPI against the development of allergen-induced airway hyperresponsiveness in guinea pigs. A single 5 mg dose of SLPI was administered at different times before antigen challenge. Hyperreactivity is assessed as the change in the histamine dose required to induce a 100% change in airway resistance (PC100) 24 hours after antigen challenge (mean± SE, n=4-10) (*p<0.05 effect of SLPI vs. antigen-stimulated response).

FIG. 3. Depicts the effect of SLPI on antigen-stimulated bronchial responses in sheep. SLPI was preadministered as a 3 mg aerosol dose daily for 3 days and 0.5 hour before antigen challenge. FIG. 3a. Early and late phase bronchoconstriction are assessed as the percent increase of specific lung resistance over an 8-hour period following antigen challenge (mean±SE, n=4) (*p<0.05 effect of SLPI vs. antigen-stimulated response). FIG. 3b. Airway hyperresponsiveness is assessed as the change in the carbachol dose required to induce a 400% change in airway resistance (PC400) 24 hours after antigen challenge (mean±SE, n=4) (*p<0.05 effect of SLPI vs. antigen-stimulated response).

FIG. 4. Depicts the effect of SLPI on antigen-induced airway responses in sheep when administered one hour after antigen challenge. SLPI was administered as a single 30 mg aerosol dose 0.5 hour before antigen challenge. FIG. 4a. Early and late phase bronchoconstriction are assessed as the percent increase of specific lung resistance over an 8-hour period following antigen challenge (mean± SE, n=5)(*p<0.05 effect of SLPI vs. antigen-stimulated response). FIG. 4b. Airway hyperresponsiveness is assessed as the change in the carbachol dose required to induce a 400% change in airway resistance (PC400) 24 hours after antigen challenge (mean±SE, n=5) (*p<0.05 effect of SLPI vs. antigen-stimulated response).

FIG. 5. Depicts the effect of SLPI on Ascaris-Stimulated Reduction of Tracheal Mucus Velocity in Sheep. Tracheal mucus velocity is assessed as a percent change of the baseline response following antigen challenge (#p<0.05 antigen-stimulated response vs. baseline values). FIG. 5a. SLPI was preadministered as a 3 mg aerosol dose daily for 3 days and 0.5 hour before antigen challenge (mean±SE, n=3) (*p<0.05 effect of SLPI vs. antigen-stimulated response). FIG. 5b. SLPI was administered as a single 30 mg aerosol dose one hour after antigen challenge (mean±SE, n=6) (*p<0.05 effect of SLPI vs. antigen-stimulated response).

FIG. 6. Depicts the effect of SLPI dry powder formulation on antigen-stimulated bronchial responses in guinea pigs. Hyperreactivity is determined as the shift of the dose-dependent bronchoconstriction (assessed as Pause_{enhanced}) to histamine evaluated 6 hours after antigen challenge (mean±SE, n=10) (#p<0.05 antigen-stimulated response vs. baseline values). Single dose intratracheal insufflation of SLPI powder or instillation of SLPI solution one hour before antigen challenge inhibits the development of hyperresponsiveness (mean±SE, n=4-6) (*p<0.05 effect of SLPI vs. antigen-stimulated response).

FIG. 7. Depicts the effect of a dry powder formulation of SLPI against antigen-induced early and late bronchoconstriction (FIG. 7a) and the evaluation of the development of airway hyperresponsiveness in sheep (FIG. 7b). SLPI was preadministered as a 3 mg dry powder aerosol dose daily for 3 days and 0.5 hour before antigen challenge. Early and late phase bronchoconstriction are assessed as the percent increase of specific lung resistance over an 8-hour period following antigen challenge (mean±SE, n=4) (*p<0.05 effect of SLPI vs. antigen-stimulated response). Airway hyperresponsiveness is assessed as the change in the carbachol dose required to induce a 400% change in airway resistance (PC400) 24 hours after antigen challenge (mean±SE, n=4) (*p<0.05 effect of SLPI vs. antigen-stimulated response).

### DETAILED DESCRIPTION AND PREFERRED EMBODIMENTS

Studies indicate that during a single breath of an aerosol compound, the efficiency of delivering aerosols to the lungs with conventional nebulizers has been relatively poor and variable among patients (Patton, et al. Respiratory Drug Delivery IV, 65-74 (1994); Coleman, et al., Annals of Pharmacotherapy, 30: 644-55 (1996)). In addition, the location of deposition in the lung depends upon (1) breath parameters such as volume of inspiration, inspiratory flow rate, breath holding prior to expiration, the lung volume at the time the bolus of medication is administered, and expiratory flow rate, (2) the size, shape and density of the aerosol particles (i.e., the medicinal compound, any carrier, and propellant), and (3) the physiological characteristics of the patient. Conventional devices and pharmaceutical formulations do not eliminate these variables and as such do not control dosage administration.

The present invention, however, provides a dry powder protein pharmaceutical composition suitable for inhalation therapy and the treatment of pulmonary conditions such as asthma. The composition is uniquely formulated and produced such that the protein therapeutic agent retains its biological activity upon deposition to the pulmonary area.

The present invention is based in part on the discovery that secretory leukocyte protease inhibitor (SLPI) protein is active as a dry powder and that the effectiveness of the powder, which is administered by inhalation, is increased by including a pharmaceutically-acceptable carrier in the pharmaceutical composition. In the preparation of a powdered composition for inhalation delivery, it is difficult to ensure a consistently high level of dispensability of the composition. For example, if only 50% of the particles making up a powder composition are dispersed, then 50% of the composition (and thus active agent) will remain undispersed and unused. This represents a significant amount of lost active agent and means that the manufacturer must take this loss into account to ensure sufficient active agent is included for delivery to a subject. Where the cost of the active agent is high, this can mean significant extra costs for the manufacturer. The present invention addresses the problem of lost active agent through improved dispensability. The present formulations have a high level of dispensability so that a greater percentage of the active agent in a unit dosage will enter the subject's lungs and less drug is lost per inhalation.

In its most basic form, the dry powder pharmaceutical composition contains SLPI protein and one or more pharmaceutically-acceptable excipients. Such excipients include a carrier agent or carrier material which may also be referred to as a bulking agent, dispersing agent or diluent. The term “pharmaceutically acceptable” refers to an excipient that can be taken into the lungs with no significant adverse toxicological effects on the lungs and does not significantly interact with the SLPI protein.

The terms “powder” and “powdered” refer to a composition that consists of finely dispersed solid particles that are relatively free flowing and capable of being dispersed, such as by an inhalation device, and subsequently inhaled by a subject so that the particles reach the lungs. Thus, the powder is administered by inhalation therapy and is said to be “respirable” and suitable for pulmonary delivery. In general, the average particle size is equal to or less than about 10 microns (μm) in diameter but greater than 2 μm. Particles which are less than 0.5 μm may be exhaled following inhalation or adhere to the walls of the mouth during the exhalation phase. Particles less than about 3 microns in diameter pass to the alveoli. Particles which are 8 microns or greater may deposit in the mouth or throat. As a result, the larger solid particles would be ingested into the gastrointestinal tract where they could be rapidly digested and inactivated.

The present particle size for metered-dose inhalants should be within the micrometer range and should be between 1 and 8 μm with a mass median diameter of between 3 and 6 μm. The importance of particle size in the efficient delivery of particles to bronchi may also be described by the following: 4-7 microns for airways, 1-5 microns for alveoli, and >7 for mouth and throat (J. Pharm. Sci., 75:433, 1986).

The present compositions are formulated so that the SLPI protein is delivered in a particle size which maximizes deposition in the large airways (trachea, bronchi, bronchioles), as opposed to the lower respiratory tract (e.g., alveoli) or mouth and throat. The particle shapes may be irregular, uniform or mixed. Preferably, the average size of the particles or agglomerates of particles ranges from about 2 to 8 microns. More preferably the particles or agglomerates of particles of the dry powder compositions of the present invention range in diameter from 3 to 6 microns. Most desirably, the average size ranges from about 3.5 to 5.5 microns. In addition, it is desirable that >90% of the particles or agglomerates of particles in the dry powder composition fall within these ranges.

The term “dry” means that the powder composition has a moisture content such that the particles or agglomerates of particles are readily dispersible in an inhalation device. This moisture content is generally below about 10% by weight (% w) water, usually below about 5% w and preferably less than about 3% w.

The terms “dispersible” or “dispensability” refer to the degree which a powder composition can be suspended in a current of a gas, such as air, so that the dispersed particles can be respired or inhaled into the lungs of a subject. For example, a dry powder composition that is only 10% dispersible means that only 10% of the mass of finely-divided particles making up the composition can be suspended for oral inhalation into the lungs; 50% dispersability means that 50% of the mass can be suspended. A standard measurement of dispensability is described below. Preferably, the dry powder pharmaceutical compositions of the present invention are about 50% to 95% dispersible. More preferably, the compositions are 70% to 95% dispersible, and most preferably 90% to 95% dispersible.

### Carrier Component

The materials which are suitable for use as a dry powder carrier are generally relatively free-flowing particulate solids, do not thicken or polymerize upon contact with water, are toxicologically innocuous when inhaled as a dispersed powder and do not significantly interact with SLPI protein in a manner that adversely affects the desired physiological action of the protein. Materials which are suitable for use as the carrier component of the present compositions include, but are not limited to, carbohydrates, amino acids and polypeptides. Preferred carriers have the following characteristics:

1. amorphous molecules capable of forming glass upon drying,

2. higher glass transition temperatures (>40° in final powder formulation),

3. possess functional groups which can replace water during dehydration,

4. pharmaceutically safe and inert, and

5. stabilize active protein drug during storage and delivery.

The amount of dry powder carrier material that is useful in the novel compositions is an amount that serves to uniformly distribute the active agent throughout the composition so that the active agent may be delivered to a patient as a uniform dosage. A carrier material may also serve to dilute the active agent to a concentration at which the active agent can provide the desired beneficial palliative or curative results while at the same time minimizing any adverse side effects that might occur from too high a concentration. In a preferred embodiment, a single pharmaceutically acceptable carrier serves as both a bulking agent and as a diluent. In a more preferred embodiment, a single pharmaceutically acceptable carrier material also serves as a dispersing agent or lubricant. Solid particles may undergo agglomeration, caking or particle growth. This may be overcome in the dry particle composition by the addition of an agent which provides slippage between particles and/or lubrication of portions of the delivery device.

The carrier component of the powdered pharmaceutical compositions of the present invention may range from about 0% to 99% by weight of the formulation. Preferably, the carrier provides 5% to 50% by weight of the formulation, and more preferably 10% to 30% by weight of the formulation.

Carbohydrate excipients that are particularly useful in this regard include the mono-, di- and polysaccharides, sugar alcohols and other polyols. Representative monosaccharides include dextrose (anhydrous and the monohydrate; also referred to as glucose and glucose monohydrate), galactose, mannitol, D-mannose, sorbitol, sorbose and the like. Representative disaccharides include lactose, maltose, sucrose, trehalose and the like. Representative trisaccharides include those such as raffinose and the like. Other carbohydrate excipients include glycerol, xylitol, xylose, raffinose, melezitose, lactitol, maltitol, trehalose, starch and cyclodextrins such as 2-hydroxypropyl-β-cyclodextrin. Each of these materials are readily available from commercial sources.

Suitable amino acid excipients include any of the naturally occurring amino acids that form a powder under standard pharmaceutical processing techniques and include the non-polar (hydrophobic) amino acids and polar (uncharged, positively charged and negatively charged) amino acids, such amino acids are of pharmaceutical grade and are generally regarded as safe (GRAS) by the U.S. Food and Drug Administration. Representative examples of non-polar amino acids include alanine, isoleucine, leucine, methionine, phenylalanine, proline, tryptophan and valine. Representative examples of polar, uncharged amino acids include cystine, glycine, glutamine, serine, threonine, and tyrosine. Representative examples of polar, positively charged amino acids include arginine, histidine and lysine. Representative examples of negatively charged amino acids include aspartic acid and glutamic acid. These amino acids are generally available from commercial sources that provide pharmaceutical-grade products such as the Aldrich Chemical Company, Inc., Milwaukee, Wis. or Sigma Chemical Company, St. Louis, Mo.

Suitable carrier materials also include mixtures of one or more carbohydrates with one or more amino acids. Generally, the combination may exhibit a ratio of about 100:1 to about 1:100 parts by weight of a suitable carbohydrate to part by weight of a suitable amino acid, preferably such ratio will be between about 5:1 to about 1:5, more preferably 1:1. An exemplary combination of this type if the combination of mannitol with glycine.

Suitable carriers may also include pharmaceutically-acceptable, polypeptides. For purposes of this application, polypeptide is meant to encompass both naturally occurring protein and artificially constructed polypeptides in which individual amino acid units are linked together through the standard peptide amide bond (the carboxyl group of one and the amino group of another). The suitable polypeptide carrier is one that can be taken into the lungs of a patient in need thereof but will have no adverse toxicological effects at the levels used. While the carrier is typically an inactive agent, the carrier may have some inherent activity as long as such activity is not antithetical to the utility of the overall composition. Therefore, it is envisioned that the active component, SLPI protein, may also serve as the carrier component of the pharmaceutical compositions of the present invention. This is particularly suitable where larger amounts of SLPI protein will be delivered.

The polypeptide carrier is generally characterized as having a molecular weight between about 1,000 and about 200,000. An example of an agent having a low molecular weight is a polyalanine having a molecular weight of about 1000. Other polypeptides in that molecular weight range which are physiologically acceptable but inactive can also be prepared. Molecules that have a molecular weight in the range of about 3000 to 6000 are also useful. Another example representative of the proteins useful in this invention include α-lactalbumin, a constituent of milk having a molecular weight of about 14,200. Another polypeptide carrier is human serum albumin, which has a molecular weight of about 69,000 (the value is given as about 69,000 in the Merck Index, Eleventh Edition and as 68,500 in Lehniger, Second edition). Typically, the molecular weight of the polypeptide carrier is from about 1000 to about 100,000 and more particularly from about 1,000 to about 70,000.

Separate materials may be used as a dispersing agent. For example surfactants such as sorbitan trioleate, oleyl alcohol, oleic acid, lecithin and corn oil have been used as dispersing agents in powder compositions. In addition, materials such as isopropyl myristate and light mineral oil have been used as lubricants.

### Secretory Leukocyte Protease Inhibitor Protein

As used in this invention, the terms “secretory leukocyte protease inhibitor” and “SLPI” refer to human SLPI protein purified from parotid secretions as well as biologically active synthetic and recombinantly produced SLPI proteins and analogs thereof as first described by Thompson et al. in U.S. Pat. No. 4,760,130 and in pending applications U.S. patent application Ser. Nos. 08/283,477 (filed Jul. 7, 1994), 07/712,354 (filed Jun. 7, 1991) and 08/279,056 (filed Jul. 22, 1994) the disclosures of each of which are hereby incorporated by reference. SLPI proteins are also described in U.S. Pat. No. 4,845,076 (Heinzel et al.), WO 96/08275 (Bayer), U.S. Pat. No. 5,618,786 (Roosdorp et al.) and EP 346 500 (Teijin) the disclosures of which are hereby incorporated by reference.

In brief, SLPI protein comprises an amino acid sequence containing at least eight cysteine residues and possessing serine protease inhibitor activity, wherein at least one active site comprises one or more amino acid sequences selected from the group consisting of:

Gln-Cys-Leu-R₂-Tyr-Lys-Lys-Pro-Glu-Cys-Gln-Ser-Asp; and
Gln-Cys-R₈-R₃-R₉-Asn-Pro-Pro-Asn-Phe-Cys-Glu-R₄-Asp

wherein R₂, R₃ and R₄ are the same or different and are selected from the group consisting of methionine, valine, alanine, phenylalanine, tyrosine, tryptophan, lysine, glycine and arginine; and R₈ and R₉ are the same or different and are selected from the group consisting of methionine, valine, alanine, phenylalanine, tyrosine, tryptophan, lysine, glycine, leucine and arginine.

Mature human SLPI has the following amino acid sequence:

Ser-Gly-Lys-Ser-Phe-Lys-Ala-Gly-Val-Cys-Pro-Pro-Lys Lys-Ser-
Ala-Gln-Cys-Leu-Arg-Tyr-Lys-Lys-Pro-Glu-Cys-Gln-Ser-Asp-Trp-
Gln-Cys-Pro-Gly-Lys-Lys-Arg-Cys-Cys-Pro-Asp-Thr-Cys-Gly-Ile-
Lys-Cys-Leu-Asp-Pro-Val-Asp-Thr-Pro-Asn-Pro-Thr-Arg-Arg-Lys-
Pro-Gly-Lys-Cys-Pro-Val-Thr-Tyr-Gly-Gln-Cys-Leu-Met-Leu-Asn-
Pro-Pro-Asn-Phe-Cys-Glu-Met-Asp-Gly-Gln-Cys-Lys-Arg-Asp-Leu-
Lys-Cys-Cys-Met-Gly-Met-Cys-Gly-Lys-Ser-Cys-Val-Ser-Pro-Val-
Lys-Ala.

Exemplary SLPI analogs include the following:

R₁-Gly-Lys-Ser-Phe-Lys-Ala-Gly-Val-Cys-Pro-Pro-Lys-Lys-Ser-
Ala-Gln-Cys-Leu-R₂-Tyr-Lys-Lys-Pro-Glu-Cys-Gln-Ser-Asp-Trp-
Gln-Cys-Pro-Gly-Lys-Lys-Arg-Cys-Cys-Pro-Asp-Thr-Cys-Gly-Ile-
Lys-Cys-Leu-Asp-Pro-Val-Asp-Thr-Pro-Asn-Pro-Thr-Arg-Arg-Lys-
Pro-Gly-Lys-Cys-Pro-Val-Thr-Tyr-Gly-Gln-Cys-R₈-R₃-R₉-Asn-
Pro-Pro-Asn-Phe-Cys-Glu-R₄-Asp-Gly-Gln-Cys-Lys-Arg-Asp-Leu-
Lys-Cys-Cys-R₅-Gly-R₆-Cys-Gly-Lys-Ser-Cys-Val-Ser-Pro-Val-
Lys-R₇

wherein R₁ and R₇ are the same or different and are selected from the group consisting of serine, alanine or a substituted amino acid residue; R₂, R₃, R₄, R₅ and R₆ are the same or different and are selected from the group consisting of methionine, valine, alanine, phenylalanine, tyrosine, tryptophan, lysine, glycine and arginine; and R₈ and R₉ are the same or different and are selected from the group consisting of methionine, valine, alanine, phenylalanine, tyrosine, tryptophan, lysine, glycine, leucine and arginine.

SLPI is a selective inhibitor of serine proteases. SLPI has been shown to inhibit tryptase, cathepsin G, elastase, chymotrypsin, chymase and trypsin, with no inhibition of kallikrein (tissue or plasma), thrombin, Factor Xa, or plasmin

By “biologically active,” it is meant that the proteins or polypeptides have substantially the same protease inhibition profile of human SLPI or a portion of the human SLPI protein. It will be appreciated by those skilled in the art that the biologically active proteins and polypeptides will have an amino acid sequence substantially homologous to that of human SLPI. “Substantially homologous”, as used herein, refers to an amino acid sequence sharing a degree of “similarity” or homology to the human SLPI protein amino acid sequence (the native serine protease inhibitor isolated from human parotid secretions) such that the homologous sequence is expected to have a biological activity or function similar to that described for human SLPI protein.

It is preferable that the degree of homology or identity is equal to or in excess of 70% (i.e., a range of from 70% to 100% homology). Thus, a preferable “substantially homologous” SLPI protein may have a degree of homology greater than or equal to 70% of the amino acid sequence of human SLPI. More preferably the degree of homology may be equal to or in excess of 80 or 85%. Even more preferably it is equal to or in excess of 90%, or most preferably it is equal to or in excess of 95%. The percentage homology or percent identity as described above is calculated as the percentage of the components found in the smaller of the two sequences being compared that may also be found in the larger of the two sequences, wherein a component is a sequence of four, contiguous amino acids.

It will be appreciated by those skilled in the art, that individual or grouped amino acid residues can be changed, positionally exchanged (e.g.s, reverse ordered or reordered) or deleted entirely in an amino acid sequence without affecting the three dimensional configuration or activity of the molecule. Thus, analogs which are useful in the practice of the present invention may have one or more amino acid additions, substitutions, and/or deletions as compared to purified, native human SLPI. One particular embodiment of an analog comprising an additional amino acid is where an initial methionine amino acid residue is present at amino acid position ⁻1. Substitution analogs may be particularly useful in that such analogs may enable greater and/or differential carbohydrate modifications as compared to naturally-derived SLPI. Such modifications are well within the ability of one skilled in the art and are also described in the above-referenced SLPI patents and applications.

Other useful SLPI analogs may have differential carbohydrate modifications, including SLPI molecules containing different patterns of glycosylation, such as the addition or deletion of one or more oligosaccharide chains, differing levels of sialation, etc. See generally Protein Glycosylation: Cellular, Biotechnical, and Analytical Aspects (1991), edited by H. S. Conradt, VCH, N.Y, N.Y.

In addition, SLPI protein derivatives may be generated. These include molecules wherein the protein is conjugated to another chemical substance, such as polyethylene glycol (PEG, see U.S. Pat. No. 4,179,337, hereby incorporated by reference). Other useful chemical conjugations may include methylation, amidation, etc. Furthermore, SLPI (or a biologically active fragment thereof) may be conjugated to another protein molecule. For example, such conjugation may be accomplished by chemical or peptide linkers. See generally Chemical Reagents for Protein Modification, 2d. Ed., R. L. Lundblad, CRC, Boca Raton, Fla., pp. 287-304, 1991. The SLPI protein may also be a chimeric protein molecule, wherein all or a portion of the primary amino acid sequence of SLPI is combined with a part or all of the primary amino acid sequence of one or more other polypeptides in a contiguous polypeptide chain. For a discussion on the generation of chimeric protein molecules, see Chemical Reagents for Protein modification, supra, which is hereby incorporated by reference.

The SLPI protein may be a native form isolated from a mammalian organism. Suitable SLPI proteins also include the products of chemical synthetic procedures and recombinant production techniques. Exemplary recombinant procedures involve host cell expression of nucleic acid sequences encoding the SLPI protein, wherein the host cell has been modified to express the protein by means of transformation, transfection or homologous recombination.

The selection of suitable host cells (e.g., bacterial, mammalian, insect, yeast, or plant cells) and methods for transformation, culture, amplification, screening and product production and purification are well known in the art. See for example, Gething and Sambrook, Nature, 293: 620-625 (1981), or alternatively, Kaufman et al., Mol. Cell. Biol., 5 (7): 1750-1759 (1985) or Howley et al., U.S. Pat. No. 4,419,446. Additional exemplary materials and methods are discussed herein. The recombinantly modified host cell is cultured under appropriate conditions, and the expressed SLPI protein is then optionally recovered, isolated and purified from a culture medium (or from the cell, if expressed intracellularly) by an appropriate means known to those skilled in the art.

Different host cells have characteristic and specific mechanisms for the translational and post-translational processing and modification (e.g., glycosylation, cleavage) of proteins. Appropriate cell lines or host systems can be chosen to ensure the desired modification and processing of the foreign protein expressed. For example, expression in a bacterial system can be used to produce an unglycosylated core protein product. Expression in yeast may be used to produce a glycosylated product. Expression in mammalian cells can be used to ensure “native” glycosylation of the protein. Furthermore, different vector/host expression systems may effect processing reactions, such as proteolytic cleavages, to different extents.

Suitable host cells for cloning or expressing the vectors disclosed herein are prokaryote, yeast, or higher eukaryote cells. Eukaryotic microbes such as filamentous fungi or yeast may be suitable hosts for the expression of SLPI proteins. Saccharomyces cerevisiae, or common baker's yeast, is the most commonly used among lower eukaryotic host microorganisms, but a number of other genera, species, and strains are well known and commonly available.

Host cells to be used for the expression of glycosylated SLPI protein are also derived from multicellular organisms. Such host cells are capable of complex processing and glycosylation activities. In principle, any higher eukaryotic cell culture might be used, whether such culture involves vertebrate or invertebrate cells, including plant and insect cells. The propagation of vertebrate cells in culture (tissue culture) is a well known procedure. Examples of useful mammalian host cell lines include, but are not limited to, monkey kidney CV1 line transformed by SV40 (COS7), human embryonic kidney line (293 or 293 cells subcloned for growth in suspension culture), baby hamster kidney cells, and Chinese hamster ovary cells. Other suitable mammalian cell lines include but are not limited to, HeLa, mouse L-929 cells, 3T3 lines derived from Swiss, Balb-c or NIH mice, BHK or HaK hamster cell lines.

Suitable host cells also include prokaryotic cells. Prokaryotic host cells include, but are not limited to, bacterial cells, such as Gram-negative or Gram-positive organisms, for example, Escherichia coli, Bacilli such as B. subtilis, Pseudomonas species such as P. aeruginosa, Salmonella typhimurium, or Serratia marcescans. For example, the various strains of E. coli (e.g., HB101, DH5a, DH10, and MC1061) are well-known as host cells in the field of biotechnology. Various strains of Streptomyces spp. and the like may also be employed. Presently preferred host cells for producing SLPI proteins are bacterial cells (e.g., E. coli) and mammalian cells (such as Chinese hamster ovary cells, COS cells, etc.)

### Other Excipients

In addition to the carrier, the pharmaceutical composition may include other pharmaceutically-acceptable excipients that may be used to facilitate delivery or enhance therapeutic action. Additional excipients include, but are not limited to, bulking agents, glass-forming agents, stabilizers, isotonic modifier, propellants, surfactants, and buffers. Other excipients which may be used in the compositions include preservatives, antioxidants, sweeteners and taste masking agents. Stabilizers include, but are not limited to, sugars such as sucrose, trehalose, mannitol, lactose, glucose, fructose, and galactose, amino acids such as glycine, lysine, glutamic acid, aspartic acid, arginine, and asparagine, proteins such as albumin and gelatin, salts such as sodium chloride, potassium chloride, and sodium sulfate, and polymers such as PVP, PEG, and PVA. These stabilizers can also be used as a glass-forming amorphous additive or as an isotonic modifier.

Buffers may be added to control the pH of formulation for the delivered protein. Buffer materials include, but are not limited to, citrate, phosphate histidine, glutamate, succinate, or acetate.

Air or various physiologically acceptable inert gases may be employed as an aerosolizing or suspending agent to suspend the dry powder particles for inhalation. Where an inert gas is employed, it will normally be present in about 0.5 to 5 weight percent. The gas or propellant may be any material conventionally employed for this purpose which does not adversely interact with the SLPI protein or human lung tissue. Suitable propellants include chlorofluorocarbon; hydrochlorofluorocarbon; hydrofluorocarbon; or a hydrocarbon, including trichlorofluoromethane, dichlorodifluoromethane, dichlorotetrafluoroethane, dichlorotetrafluoroethanol, and 1,1,1,2-tetrafluoroethane, or combinations thereof. The propellant may also be substantially free of chlorofluorocarbons for oral and/or nasal administration, see for example the non-chlorofluorocarbon aerosol formulations as described in U.S. Pat. No. 5,474,759 (Fassberg et al.). A preferred suspending agent is air.

The properly sized particles may be suspended in a propellant with or without the aid of a surfactant. The composition may also include a surfactant to stabilize protein under the shear-stress of spray-drying, to improve the physical properties of the powder, and to enhance delivery of protein to the airway surface. Suitable surfactants include, but are not limited to, fatty acids, phospholipids, sorbitan trioleate, soya lecithin, oleic acid, Polysorbates, Poloxamer, Briji and Polyoxyl stearate.

In addition, physiologically acceptable surfactants may include glycerides, and more particularly diglycerides, where one of the carboxylic acids is of from 2 to 4 carbon atoms, and the other will be of from 12 to 20 carbon atoms, more usually of from 16 to 18 carbon atoms, either saturated or unsaturated. The surfactant may vary from about 0.01 to 10 weight percent of the formulation.

The compositions of the present invention may also include an excipient which serves to enhance the absorption of the serine protease inhibitor protein through the layer of epithelial cells in the lower respiratory tract, and into the adjacent pulmonary vasculature. The enhancer can accomplish this by any of several possible mechanisms:

(1) enhancement of the paracellular permeability of the active agent by inducing structural changes in the tight junctions between the epithelial cells.

(2) enhancement of the transcellular permeability of the active agent by interacting with or extracting protein or lipid constituents of the membrane, and thereby perturbing the membrane's integrity.

(3) interaction between enhancer and the active agent which increases the solubility of the active agent in aqueous solution. This may occur by preventing formation of aggregates (dimers, trimers, hexamers), or by solubilizing the active agent molecules in enhancer micelles.

(4) decreasing the viscosity of, or dissolving, the mucus barrier lining the alveoli and passages of the lung, thereby exposing the epithelial surface for direct absorption of the active agent.

Enhancers may function by one or more of the mechanisms set forth above. An enhancer which acts by several mechanisms is more likely to promote efficient absorption of the active agent than one which employs only one or two of the mechanisms. For example, surfactants may serve as enhancers and are believed to act by all four mechanisms listed above. Surfactants are amphiphilic molecules having both a lipophilic and a hydrophilic moiety, with varying balance between these two characteristics. If the molecule is very lipophilic, the low solubility of the substance in water may limit its usefulness. If the hydrophilic part overwhelmingly dominates, however, the surface active properties of the molecule may be minimal. To be effective, therefore, the surfactant must strike an appropriate balance between sufficient solubility and sufficient surface activity. The use of such enhancers is described in U.S. Pat. No. 5,518,998 (Backstrom et al.) which is herein incorporated by reference.

An excipient may serve as an enhancer if the amount of protein absorbed into the subepithelial space in the presence of the enhancer is higher than the amount absorbed in the absence of enhancer. Such enhancement would improve efficacy through inhibition of proteases in tissue compartments other than the lumen of the airway. Exemplary materials which may be used to enhance absorption include, but are not limited to, sodium; potassium; phospholipids; acylcarnitines; sodium salts of ursodeoxycholate, taurocholate, glycocholate; and taurodihydrofusidate.

Additional potentially useful enhancers are sodium salicylate, sodium 5-methoxysalicylate, naturally occurring surfactants such as salts of glycyrrhizine acid, saponin glycosides and acyl carnitines; sodium salts of saturated fatty acids of carbon chain length 10 (i.e., sodium caprate), 12 (sodium laurate) and 14 (sodium myristate); and potassium and lysine salts of capric acid (if the carbon chain length is shorter than about 10, the surface activity of the surfactant may be too low, and if the chain length is longer than about 14, decreased solubility of the fatty acid salt in water limits its usefulness); phospholipids such as lysophospatidylcholine; alkyl glycosides such as octylglucopyranoside, thioglucopyranosides and maltopyranosides; and cyclodextrins and derivatives thereof.

### Combinations

The novel dry powder pharmaceutical compositions may also optionally include an active agent or agent in addition to the SLPI protein. For example, the composition may include a bronchodilator compound or anti-inflammatory agent. Such a compound could be any compound effective in counteracting bronchoconstriction or the development of airway hyperresponsiveness. Types of drugs known to be useful in the inhalation treatment of asthma include respiratory NSAIDs (cromolyn sodium, nedocromil, etc.); anticholinergic agents (such as atropine and ipratropium bromide); beta 2 agonists (such as adrenaline, isoproterenol, ephedrine, salbutamol, terbutaline, orciprenaline, fenoterol, and isoetharine), methylxanthines (such as theophylline); calcium-channel blockers (such as verapamil); and glucocorticoids (such as prednisone, prednisolone, dexamethasone, beclomethasone dipropionate, and beclomethasone valerate), as described in Chapter 39 of Principles of Medical Pharmacology, Fifth Edition, Kalant and Roschlau, Ed. (B. C. Decker Inc., Philadelphia, 1989), herein incorporated by reference. The use and dosage of these and other effective bronchodilator drugs in inhalation therapy are well known to practitioners who routinely treat asthmatic patients.

Other suitable compounds for the preparation of combination compositions include inhibitors of TNFα, inhibitors of IgE synthesis or activity, inhibitors of cytokines or chemokines associated with asthma pathogenesis, other protease inhibitors, and heparin. Additional agents which can be used in combination with SLPI include monoclonal antibodies, soluble receptors, natural protein or peptide inhibitors, and medicinal chemistry-derived synthetic inhibitors.

The novel pharmaceutical compositions may also be formulated such that the additional active agent or agents have a particle size which differs from that of the SLPI protein/carrier particle or agglomerates of particles. For example, the additional active agent may have a particle size which results in the deposition of the agent in the alveoli following inhalation such that the agent exerts its effects in or is preferably absorbed from that area of the lungs.

### Uses

The compositions of the present invention are advantageously formulated for treating diseases or conditions affecting the lungs. The dry powder compositions are prepared to provide a physiologically or therapeutically effective dosage of the protein in the lungs. It has been determined that the proteins are retained in the lung epithelial lining fluid, so as to maintain an effective concentration in the lung, in contact with lung tissue, for extended periods of time. The protein thereby provides for the regulation of protease tone in the airways, the inhibition of the priming effects of proteases on stimulated effect or cell function, and the prevention of cell and tissue responses to chronic protease exposure.

In particular, the novel pharmaceutical compositions of the present invention have been found to decrease mucous production/secretion, increase mucous velocity in the airways, decrease airway hyperresponsiveness to antigen/stimulus (decreases smooth muscle contraction) and inhibit pathological changes to airway cells/tissue. The compositions may be useful for preventing (if given prior to the onset of symptoms) or reversing acute pulmonary vasoconstriction, such as may result from pneumonia, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of the newborn, perinatal aspiration syndrome, hyaline membrane disease, acute pulmonary thromboembolism, heparin-protamine reactions, sepsis, asthma, status asthmaticus, or hypoxia (including that which may occur during one-lung anesthesia). In addition, the compositions may be used in those cases of chronic pulmonary vasoconstriction which have a reversible component, such as may result from chronic pulmonary hypertension, bronchopulmonary dysplasia, chronic pulmonary thromboembolism, idiopathic or primary pulmonary hypertension, or chronic hypoxia. The compositions may also be used to inhibit the infectivity of respiratory viruses. Such inhibition would prevent viral-induced hyperreactivity of the airways (Tokyo Tanabe WO97/03694).

The term “therapeutically effective amount” refers to an amount of the active agent present in the powder compositions that is needed to provide the desired level of the active agent to a subject to be treated to provide the anticipated physiological response. With respect to a patient suffering from bronchoconstriction, a therapeutically effective amount of a dry powder SLPI composition may be an amount which reduces the patient's airway resistance by 20% or more, as measured by standard methods of pulmonary mechanics. For example, for a patient suffering from pulmonary vasoconstriction, a “therapeutically effective” amount of the composition may be determined as an amount which can induce either or both of the following: (1) prevention of the onset of pulmonary vasoconstriction following an injury (such as aspiration or trauma) that could be expected to result in pulmonary vasoconstriction or (2) a 20% or more decrease in the patient's DELTA PVR (the difference between the patient's elevated PVR and “normal” PVR, with normal PVR assumed to be below 1 mmHg×min/liter for an adult human, unless found to be otherwise for a given patient).

Thus, what constitutes a therapeutically effective amount of SLPI will depend on the particular disease state or condition being treated. For instance, in the case of asthma, a therapeutically effective amount of SLPI will be an amount sufficient to inhibit bronchoconstriction and development of airway hyperreactivity to provide effective reduction of asthma symptomology. The therapeutically effective amount will depend on a variety of factors which the knowledgeable practitioner will take into account in arriving at the desired dosage regimen, including the severity of the condition or illness being treated, the degree of pulmonary dysfunction, the physical condition of the subject, and so forth. In general, a dosage regimen will be followed such that the pulmonary mechanics for the individual undergoing treatment is restored.

### Pharmaceutical Composition Preparation

The present invention provides for the delivery of a dry powder SLPI composition which can be dispersed as an aerosol suitable for inhalation therapy. In brief, the pharmaceutical composition may be prepared by (a) forming a homogeneous composition containing SLPI and a pharmaceutically acceptable excipient in a solvent, (b) removing the solvent from the mixture to form a solid and (c) transforming the resulting solid into a respirable powdered pharmaceutical composition.

Another, more specific, aspect of this invention is a method for preparing a spray-dried, dispersible powdered SLPI pharmaceutical composition that comprises spray drying a homogeneous mixture of SLPI and one or more pharmaceutically acceptable carrier agents contained in a solvent. The spray-drying process is performed under conditions to provide a dispersible powdered pharmaceutical composition containing particles and/or agglomerates of particles which have a particle size ranging from less than about ten microns and greater than 3 microns which are suitable for inhalation delivery to the large airways. If delivery to the alveoli is required (e.g., emphysema) a diameter of <3 microns is desired.

The dry powder compositions may be made by vacuum concentration, open drying, freeze drying or other means of drying. For example, the process may involve the formation of an aqueous composition which is lyophilized under standard lyophilizing conditions to remove the water. The resulting solid composition is transformed into a powder by comminuting the solid by a means such as ball-milling or jet-milling to obtain a particle size which is respirable and suitable for inhalation therapy. The spray drying process, however, provides particles which have a uniform particle size without the need to perform additional manufacturing steps such as grinding, milling or micronization. It has also been found that this preferred method of producing the dry powder compositions of the present invention results in the formation of particles which do not agglomerate during storage. Because the particle size remains constant during storage, delivery methods or pre-delivery preparation do not require the removal or break-up of agglomerates.

Suitable solvents for use in the spray drying process include, but are not limited to, water, ethanol, tertiary butyl alcohol, and acetone. In the preparation of an aqueous mixture, a solution or stable suspension is formed by dissolving or suspending the active agent in water with or without a carrier excipient. The order in which the components of a composition are added is not of major significance, and while the homogenous mixture may be a solution or suspension, it is preferably a solution. The proportion of components in the aqueous mixture is consistent with the proportions that are desired in the resulting powdered composition.

The amount of SLPI protein which is employed will usually vary from about 10 to 100% by weight of the final composition. More usually the composition contains 50 to 100% by weight of SLPI protein.

The carrier component, as described in detail above, will vary from about 0% to 90% by weight of the final composition. More usually the carrier component provides about 0 to 50 weight percent of the final composition, and most preferably 10 to 30 weight percent.

One especially preferred carrier material is trehalose. At a trehalose concentration of higher than 25% (w/w), the particles have a tendency to agglomerate during the spray-drying process. It was discovered, however, that the final particle size does not deviate from the desired 3-6 micron range. Instead, the particles shrink at a higher sugar concentration, and the agglomeration of particles results in a final particle size within the optimal range. In addition, further agglomeration does not occur during storage after spray-drying.

It will be appreciated that the amount of SLPI protein employed will vary depending upon a number of factors, including the size of the particle, the desired frequency of administration, the nature of the disease, whether the treatment is for therapeutic or prophylactic purposes, etc. The period of treatment will vary widely, depending upon the therapeutic dosage, the concentration of the drug, the rate of administration, and the like. For example, a single administration or repeated administrations may be required depending upon the delivery device used. Thus, the aerosol may be administered one or more times at intervals from about 6 to 24 hours.

As described above, particle size determines the site where the drug deposits following inhalation. It has been found that particles having a size of less than about 3 μm reach the alveoli, particles less than about 0.5 μm may be exhaled following inhalation, and particles greater than about 7 μm deposit around mouth. By controlling the particle size during formulation of the pharmaceutical composition (e.g., optimizing the spray-drying conditions) the size of the particles can be optimized to have an average diameter of 5 μm in a range of between 3-6 μm for maximum deposit in the airways (bronchi) as opposed to either mouth or lung. The compositions of the present invention may also be prepared such that the majority of the final composition consists of particles having a mean particle size of from about 3.5 to 6.5 microns. Preferably, about 75% or more of the particles have a diameter in this range, and most preferably about 95% or more of the preparation mass consists of a distribution of particles having a mean particle size of 3.9 to 5.4 microns in diameter. Preferably, the final composition consists of particles having a mean particle size of from about 4.5 to 5.5 microns. The standard deviation of particle diameter for the spray-dried formulations was found to be 0.18-0.28, suggesting that the particle size is well controlled in the production process using the present protein formulation.

The dry powder formulations of the present invention also provide compositions which provide higher protein deliverability than provided by an aerosol from a liquid formulation. As a result, more protein can be delivered per puff or inhalation. In addition, the dry powder formulations have improved storage stability as compared to the prior liquid formulations. For example, the purity of SLPI decreased to less than 50% by HPLC analyses when the protein was stored in solution for 11 days at 56° C. although approximately 95% purity was maintained.

### Nebulizer SLPI Formulation

SLPI formulations suitable for use with a nebulizer, either jet or ultrasonic, contain SLPI dissolved in water or buffer at a concentration of, e.g., up to 25 mg of SLPI per mL of solution. The formulation may also include a buffer and possibly a simple sugar (e.g., for protein stabilization and regulation of osmotic pressure). Examples of buffers which may be used are sodium acetate, citrate and glycine. Preferably, the buffer will have a composition and molarity suitable to adjust the solution to a pH in the range of 5 to 7. Generally, buffer molarities of from 2 mM to 50 mM are suitable for this purpose. Examples of sugars which can be utilized are lactose, maltose, mannitol, sorbitol, trehalose, and xylose, usually in amounts ranging from 1% to 10% by weight of the formulation.

An exemplary liquid formulation suitable for delivery by means of a nebulizer contains a solution of SLPI 25 mg/mL in water, in 20 mM sodium phosphate buffer at pH 7.2, made isotonic with 39 g/L mannitol. A typical liquid formulation which is aerosolized by means of a nebulizer contained a solution (1-5 ml) of SLPI in phosphate-buffered saline, see Vogelmeier et al., Journal of Applied Physiology, 69(5):1843-1848, 1990. As described therein, 55% of the resulting aerosol droplets had a diameter of less than 3 μm and were deposited in the alveoli.

It has been found that the SLPI liquid formulations must be stored at 2° to 8° C. Tests indicate that the purity and activity of SLPI does not change in the liquid formulation for 17 months at −20° C. or 6 months at 2° to 8° C. However, exposure of the material to temperatures above 2° to 8° C., except during administration, is not recommended and may result in a loss of activity.

Two examples of commercially available nebulizers suitable for delivering such compositions are the Ultravent nebulizer, manufactured by Mallinckrodt, Inc., St. Louis, Mo., the Acorn II nebulizer, manufactured by Marquest Medical Products, Englewood, Colo., and the AERx™ pulmonary drug delivery system manufactured by Aradigm, Hayward, Calif.

### Delivery Devices

Devices capable of depositing aerosolized dry powder SLPI formulations in the airway of a patient include metered dose inhalers and powder inhalers. All such devices require the use of formulations suitable for the dispensing the active agent in an aerosol. Typically, each formulation is specific to the type of device employed and may involve the use of an appropriate propellant material, in addition to carriers or other materials.

As those skilled in the art will recognize, the operating conditions for delivery of a suitable inhalation dose may vary according to the type of mechanical device employed. For some aerosol delivery systems, such as nebulizers, the frequency of administration and operating period is dictated chiefly by the amount of therapeutic agent per unit volume in the aerosol. Some devices, such as metered dose inhalers, may produce higher aerosol concentrations than others and thus will be operated for shorter periods to give the desired result.

Devices such as powder inhalers are designed to be used until a given charge of active material is exhausted from the device. The charge loaded into the device will be formulated accordingly to contain the proper inhalation dose amount of active agent for delivery in a single administration.

The pharmaceutical compositions also may be delivered from a unit dosage receptacle containing an amount of the composition that will be sufficient to provide the desired physiological effect upon inhalation by a subject. For example, the dosage may be dispersed in a chamber that has an internal volume sufficient to capture substantially all of the powder dispersion resulting from the unit dosage receptacle. Typically, the volume of the chamber will be from about 50 ml to about 1000 ml, preferably from about 100 ml to about 750 ml. Thus, the unit dosage amount will be from about 2 mg of powder to about 20 mg of powder preferably about 4 mg to about 10 mg of powder per unit dosage. Typically, about 5 mg per unit dosage is quite effective. A preferred unit dosage receptacle is a blister pack, generally provided as a series of blister pack strips. The general process for preparing such blister packs or blister pack strips is generally known to one of skill in the art from such publications as Remington's Pharmaceutical Sciences (18th Edition) or other similar publications. The volume of such a dosage form receptacle to accommodate the needed amount of powder of this invention will be about 1 ml to about 30 ml, preferably about 2 ml to about 10 ml.

Examples of devices suitable for administering a powdered SLPI composition of the present invention include the Spinhaler™ powder inhaler (manufactured by Fisons Corp., Bedford, Mass.), the Rotahaler™ powder inhaler, the Diskhaler™ powder inhaler, and the Turbohaler™ powder inhaler devices or the like as described in “Respiratory Drug Delivery” edited by P. R. Byron, published by CRC Press, 1990, p.169. Additional devices for administering powdered compositions are described in WO 96/32096 (PCT/U.S.96/05265, filed Apr. 15, 1996, Inhale Therapeutic Systems, Palo Alto, Calif.) and U.S. Pat. No. 5,626,871 (issued May 6, 1997, Teijin Limited), the disclosures of which are incorporated herein by reference. Additional devices are exemplified by those used by Dura Pharmaceuticals, Inc., San Diego, Calif., and Glaxo Inc., Research Triangle Park, N.C.

### EXAMPLES

The following examples are offered by way of illustration and not by way of limitation.

### Example 1

### Pharmaceutical Compositions

Preparation

Exemplary SLPI powder formulations were made with the following carriers (Table 1). The powders were stored at 4° C., 29° C. and 50° C. for ten weeks.

TABLE 1
Compositions

| Carrier | SLPI:carrier |
|---|---|
| None | 100:0 |
| Trehalose | 75:25 |
| | 50:50 |
| Mannitol | 90:10 |
| | 85:15 |
| | 75:25 |
| Sucrose | 90:10 |
| | 85:15 |
| | 75:25 |

Analysis

Samples were removed for bi-weekly analysis by the following methods: size exclusion chromatography, reverse phase chromatography, cation exchange chromatography, and sodium dodecyl sulfate-polyacrylamide gel electrophoresis. In vitro bioactivity was tested using an anti-chymotrypsin assay, as described in Schonbaum et al. (J. Biol. Chem. 236, 2930-2935, (1961)).

The SLPI powder formulations were characterized by particle size and moisture content. These data are summarized in Table 2.

TABLE 2
Formulation Characteristics

| | | Moisture Content |
|---|---|---|
| Sample | Particle Diameter (μm) | (wt/wt %) |
| SLPI | 3.90 ± 0.28 | 8.74 ± 0.36 |
| +mannitol 10% | 5.44 ± 0.14 | 5.76 ± 0.39 |
| +mannitol 15% | 5.22 ± 0.27 | 5.25 ± 0.11 |
| +mannitol 25% | 6.61 ± 0.67 | 6.02 ± 0.37 |
| +sucrose 10% | 5.57 ± 0.09 | 6.97 ± 0.65 |
| +sucrose 15% | 5.14 ± 0.27 | 5.07 ± 0.68 |
| +sucrose 25% | 5.69 ± 0.40 | 5.90 ± 0.55 |
| +trehalose 25% | 5.40 ± 0.18 | 6.55 ± 0.30 |
| +trehalose 50% | 5.36 ± 0.24 | 3.31 ± 0.22 |

As demonstrated by these results, the particle sizes of the spray-dried powder compositions fall within a desired range of 3.5 to 6 microns which is optimal for delivery to the large airways (bronchial delivery) rather than for deposition in the alveolar region, mouth or throat.

In addition, the inclusion of the sugars stabilizes the SLPI protein in the spray-dried powder. In the presence of 25% (w/w) or higher of trehalose, SLPI is stable at room temperature for two years. Mannitol or sucrose also provide for SLPI stability for two years at room temperature. This enhanced stability and form provides for compositions than can be advantageously packaged and stored in such containers and devices as blister packs, metered-dose inhalers and dry powder inhalers as described above.

Therefore, the compositions provide distinct advantages over the previous liquid formulations which utilize a nebulizer for delivery. The nebulizers are not usually portable due to size and requirements for external power supplies or compressed air supplies. In addition, the spray-dried powder composition avoids the limitations of inefficient delivery, wide ranges of particle size and extended time periods for the administration of a single dose of a liquid formulation.

A variety of protein-based protease inhibitors, and variants/mutants thereof, may be formulated as dry powder compositions similar to those described for SLPI. Such pharmaceutical compositions may include the following protease inhibitors in combination with one or more of the indicated carriers.

TABLE 3
Exemplary Components for
Protease Inhibitor Dry Powder Pharmaceutical Compositions

| | Selected Carriers |
|---|---|
| Protease Inhibitor | (e.g., 10-90% by weight) |
| alpha-1 antitrypsin | mannitol |
| leech-derived tryptase inhibitor | sucrose |
| soy bean trypsin inhibitor | trehalose |
| aprotinin | galactose |
| alpha-1 antichymotrypsin | D-mannose |
| kallistatin | sorbitol |
| ecotin | sorbose |
| alpha 2-macroglobulin | lactose |
| alpha 2-antiplasmin | maltose |
| C-reactive protein | raffinose |
| bronchial mucous inhibitor | glycerol |
| C-1-inhibitor | xylitol |
| cystatin | xylose |
| beta 1-antigellagenase | raffinose |
| serine amyloid A protein | melezitose |
| alpha cysteine protease inhibitors | lactitol |
| inter-alpha-trypsin inhibitor | maltitol |
| tissue inhibitors of | starch |
| metalloproteinases (TIMP 1,2) | 2-hydroxypropyl-β-cyclodextrin |
| | α-lactalbumin |
| | human serum albumin |
| | polyalanine |
| | non-polar (hydrophobic) amino acids |
| | polar (uncharged, positively charged |
| | and negatively charged) amino acids |
| | charged oligosaccharides (heparin, |
| | dextran sulfate, etc.) |

The protease inhibitor dry powder pharmaceutical compositions are useful as anti-inflammatory agents, in particular, those compositions containing protease inhibitors which have a specific activity for mast cell mediators or the proteases derived therefrom. The compositions are most suitable for inhalation and topical use, preferably administered at the site of inflammation. The treatment can be simultaneous with or followed with the addition of other therapeutic agents, for example, an appropriate steroid or antibiotic. SLPI dry powder pharmaceutical compositions are advantageously used in the treatment of pulmonary conditions such as asthma. In particular, the SLPI dry powder pharmaceutical compositions may be used to inhibit pulmonary mucous production/secretion, increase mucous velocity in the airway, decrease airway hyperresponsiveness to antigen/stimulus and inhibit pathological changes to airway cells/tissue. The SLPI compositions may also be used in the treatment of viral infections as disclosed in patent application Ser. No. 08/483,503 which is incorporated by reference herein

### Example 2

### Spray Dried Powder SLPI Compositions

An aqueous mixture is prepared at a temperature that is above the freezing point of water but below a temperature which will adversely affect the activity of the active agent(s). Generally the temperature will be between about 20-30° C., preferably at ambient temperatures. The pH of the solution can be adjusted by including a buffering material which will be appropriate for the desired stability of the active agent. The pH will generally be in the neutral range of about pH 6-8, preferably about pH 7. Suitable buffering compositions can include a citrate-base buffer, phosphate-base buffer or an acetate-base buffer. Other excipients may be included in the aqueous composition which would enhance the stability or the suspendability of the mixtures while in aqueous form. Typically, the aqueous solution is formed simply by mixing the appropriate concentrations of materials in water with stirring until all the materials are dissolved or dispersed and suspended in the water.

Preferably, the water removal and transformation to a powder take place in a spray drying environment which allows the two steps to take place at the same time. This method involves bringing together a highly dispersed liquid, which is an aqueous composition as described above, and a sufficient volume of hot air to produce evaporation and drying of the liquid droplets. The feed liquid may be a solution, slurry, emulsion, gel or paste provided the feed is capable of being atomized. Preferably, a solution is employed. The feed material is sprayed into a current of warm filtered air that evaporates the water and conveys the dried product to a collector. The spent air is then exhausted with the moisture. Typically, the resulting spray-dried powdered particles are homogenous, approximately spherical in shape and nearly uniform in size. A further discussion of spray drying can be found in Chapter 89 of Remington's at pages 1646-1647.

Generally the inlet temperature and the outlet temperature of the spray dry equipment are not critical but will be of such a level to provide the desired particle size and to result in a product that has the desired activity of the active agent. The inlet temperature may be between temperatures of 80° C. to about 150° C. with the outlet temperature being at temperatures of about 50° C. to 100° C. Preferably these temperatures will be from 90° C. to 120° C. for inlet and from 60° C. to 90° C. for the outlet. The flow rate which is used in the spray drying equipment generally will be about 3 ml per minute to about 5 ml per minute. The atomizer air flow rate will vary between values of 700 LPH (liters per hour) to about 800 LPH. Secondary drying is not needed, but may be employed.

The particle size distribution (PSD) of the powder composition may be measured using an Horiba CAPA-700 centrifugal sedimentation particle size analyzer. A measurement may be taken on approximately 5 mg of powder that is suspended in approximately 5 ml of Sedisperse A-11 (Micromeritics, Norcross, Ga.) and briefly sonicated before analysis. The instrument is configured to measure a particle size range of 0.40 to 10 μm in diameter and the centrifuge is operated at 2000 rpm. The particle size distribution of the powder is characterized by mass median diameter (MMD).

### Example 3

### Dispensability

To determine the dispensability or dispersibility of the resulting pharmaceutical composition as compared to other compositions, such as liquid droplet aerosols, one can quantify the deliverable dose of a unit dosage form by aerosolizing the powder composition, collecting the aerosolized composition and measuring the delivered material using the equipment and procedure as described hereinafter.

A high level of dispensability leads to a high percentage of delivered dose of the composition. Delivered dose is a key parameter in the success of a powdered composition. The efficiency by which a composition is delivered by a dry powder pulmonary inhaler device may be measured by (1) aerosolizing the fine particle powder in an aerosol chamber, and (2) delivering those fine particles through the mouthpiece of a device during a test inhalation. For example, the dose delivered with each formulation may be determined as follows. The device is actuated, suspending the powder in the device's aerosol chamber. The suspended particles are then drawn from the chamber at a determined rate (e.g., an air flow rate of about 30 L/min for 2.5 seconds (1.25 L inspired volume)) and a sample is collected on a suitable filter (e.g., a polyvinylidene fluoride membrane filter with a 0.65 μm pore size may be particularly useful). The sampling airflow pattern may be controlled by an automatic timer and operated to simulate a patient's slow deep inspiration. The overall efficiency (delivered dose) and percent of the powder left in the aerosol chamber after actuation may be determined gravimetrically by weighing the powder on the filter and the amount of powder remaining in a storage chamber such as a blister pack.

The extent of dispensability may be determined as follows:

1. total mass of powdered composition in a unit dosage (e.g., a 5 mg blister pack

2. total mass of powdered composition aerosolized in a unit dosage and collected on filter (e.g., 2.5 mg)

3. dispensability is defined as the mass of powder collected on filter divided by the mass of powder in the blister pack expressed as a percent (e.g., 2.5÷5= 50%). Equipment which may be used in determining dispensability is described in WO 93/00951 (published Jan. 21, 1993, entitled Method and Device For Aerosolized Medicaments) which is incorporated herein by reference.

### Example 4

### Effects of SLPI on Antigen-induced Pulmonary Responses and Pathologic Changes

Secretory leukocyte protease inhibitor (SLPI) is a naturally occurring protein of the human airway which exhibits broad spectrum inhibitory activity against mast cell and leukocyte serine proteases implicated in the pathogenesis of asthma. To assess the potential therapeutic utility of SLPI in asthma, its effects on antigen-induced pulmonary responses, as well as pathologic changes of the airways associated with asthma, were evaluated. SLPI inhibited early and late phase bronchoconstriction in sheep and the development of airway hyperreactivity in guinea pigs and sheep. Rapid onset of action and prolonged pharmacodynamic activity of SLPI were observed. In addition, SLPI inhibited the antigen-induced decrease of tracheal mucus velocity in sheep. These results provide evidence that pulmonary SLPI delivery is suitable for therapeutic intervention against the pathophysiology of asthma as well as its underlying pathology.

Asthma is a chronic pulmonary disorder characterized by two key pathophysiologic components: recurrent bronchoconstriction and development of airway hyperresponsiveness to allergic and environmental stimuli. These physiologic responses are manifest as cough, wheezing, and shortness of breath (National Asthma Education and Prevention Program. Expert panel report II: Guidelines for the diagnosis and management of asthma. 1997). While there has been great success in the development of symptomatic therapies for asthma, the concept that these pathophysiologic responses occur within airways that have been profoundly modified has not been fully addressed. Such pathologic changes of the airways include bronchial infiltration of inflammatory cells, mucus gland hypertrophy and mucus hypersecretion, epithelial cell desquamation, fibrosis, edema, and smooth muscle hypertrophy (Dunnill, M. S. J. Clin. Pathol. 13:27-33, 1960.) Despite the various therapeutic approaches available, asthma continues to represent a significant unmet medical need, particularly for patients with moderate and severe asthma. The population of patients with severe asthma continues to grow and the rate of hospitalization among patients with asthma remains high. It has been hypothesized that current therapies fail to address a fundamental component of asthma pathogenesis.

Emerging evidence suggests that serine proteases play a key role in the pathogenesis of asthma (Caughey, G. Am. J. Physiol. (Lung Cell. Mol. Physiol.), 257:L39-L46, 1989; Walls, A. F. 1994. Asthma and Rhinitis, 801-824, edited by Busse, W. W. and S. T. Holgate. Boston: Blackwell Scientific Publications). Mast cell and leukocyte serine proteases are elevated in the airways of asthmatic patients (Wenzel, et al. Am. Rev. Respir. Dis., 137:1002-1008, 1988; Broide, et al. J. Allergy Clin. Immunol., 88:637-648, 1991; Fahy, et al. J Allergy Clin. Immunol., 95:843-852, 1995). In addition, patients with reduced anti-protease activity as a result of α-1-antitrypsin deficiency have an increased propensity to develop asthma (Eden, et al. Am. J. Respir. Crit. Care Med., 156:68-74, 1997). In animal studies, instillation of elastase (Suzuki, et al. Am. J. Respir. Crit. Care Med., 153:1405-1411, 1996) or tryptase (Molinari, et al. Am. J Respir. Crit. Care Med., 154:649-653, 1996) promotes bronchoconstriction and the development of airway hyperresponsiveness, while specific inhibitors of these proteases reduce antigen-induced airway responses in vivo (Fujimoto, et al. Respiration Physiol., 100:91-100, 1994; Clark, et al. Am. J. Respir. Crit. Care Med., 152:2076-2083, 1995). Serine proteases, including cathepsin G (Fahy, et al. Am. Rev. Respir. Dis., 146:1430-1433, 1992; Venaille, et al. J. Allergy Clin. Immunol., 95:597-606, 1995), elastase (Mendis, et al. Immunol. Cell Biol., 68:95-105, 1990), and tryptase (Ruoss, et al. J. Clin. Invest., 88:493-499, 1991; Brown, et al. Am. J. Respir. Cell Mol. Biol., 13:227-236, 1995; Imamura, et al. Lab Invest., 74:861-870, 1996; Walls, et al. Int. Arch. Allergy Immunol., 107:372-373, 1995) have also been implicated in promoting airway pathology associated with asthma. In addition, tryptase stimulates allergic mediator release from mast cells (He, et al. Eur. J. Pharmacol., 328:89-97, 1997). These observations support the contribution of serine proteases to both the pathophysiology and airway pathology associated with asthma and indicate that the inhibition of mast cell and leukocyte serine proteases represent an important new approach for the treatment of asthma.

SLPI is a naturally occurring protease inhibitor produced by mucosal epithelial cells, serous cells, and bronchiolar goblet cells in human airways (Thompson, R. C., and K. Ohlsson. Proc. Natl. Acad. Sci. USA. 83:6692-6696, 1986; Eisenberg, et al. J. Biol. Chem. 265: 7976-7981, 1990; Vogelmeier, et al. Clin. Invest. 87:482-488, 1991). SLPI exhibits potent broad spectrum inhibition of mast cell and leukocyte serine proteases. In addition, physical properties of this 11.7 kDa, non-glycosylated protein contribute to its application in the treatment of inflammatory pulmonary diseases (Vogelmeier, et al. J. Appl. Physiol. 69:1843-1848, 1990). Acid stability of SLPI allows the inhibitor to remain functionally active under acidic inflammatory conditions. With a pI>9, SLPI may also bind tissue sites favored by proteases, thus facilitating prolonged inhibition of protease activity in the bronchi. In addition, the N-terminal domain of SLPI provides for interaction with heparin to accelerate binding of the inhibitor to serine proteases (Faller, et al. Biochemistry 31:8285-8290, 1992). Based on its biochemical profile, the following studies were conducted to evaluate the efficacy of SLPI against the pathophysiology and pathology associated with asthma.

### METHODS

### Protein

Recombinant SLPI was expressed and purified as previously described (Eisenberg, et al. J. Biol. Chem. 265: 7976-7981, 1990). The recombinant protein was >99% pure as assessed by sodium dodecyl sulfate polyacrylamide gel electrophoresis and high performance liquid chromatography. The purified protein contained <0.72 EU lipopolysaccharide/mg protein.

### Biochemical Assays

Human lung tryptase (Cortex Biochem, Inc., San Leandro, Calif.) activity was assessed using vasoactive intestinal peptide (VIP) (Sigma Chemical Co., St. Louis, Mo.) as a substrate in 100 mM Tris—HCl (pH 8.0) with 1 μg/ml heparin and 0.02% Triton X-100. Tryptase was incubated with various concentrations of SLPI for one hour at 37° C. VIP cleavage was assessed by reverse phase high performance liquid chromatography (Delaria, K. and D. Muller. Anal. Biochem., 236:74-81, 1996). The Kᵢ value was determined from measurements of fractional activity of tryptase at various SLPI concentrations.

Other serine proteases were assayed using specific chromogenic peptide-p-nitroanilide (pNA) substrates in a 96-well microtiter plate format. Each protease was incubated with various concentrations of SLPI for 15 minutes at 37° C. in specific assay buffer. The residual protease activity was measured following addition of the respective substrate. The p-nitroaniline product of proteolysis was quantified at 405 nm on a SpectraMAX 340 plate reader (Molecular Devices, Sunnyvale, Calif.). Human neutrophil elastase (Calbiochem-Novabiochem International, San Diego, Calif.) was assayed using pyroGlu-Pro-Val-pNA (Pharmacia Hepar Inc., Franklin, Ohio) in 100 mM Tris—HCl, pH 8.3, 0.96 M NaCl, 1% BSA (Kramps, et al., Scand. J. Clin. Lab. Invest., 43:427-432, 1983). Bovine pancreatic trypsin (TPCK-treated) (Sigma) was assayed using N-α-Benzoyl-L-Arg-pNA (Boehringer Mannheim Corp., Indianapolis, Ind.) in 50 mM Tris—HCl, pH 8.2, 20 mM CaCl₂ (Somorin, et al., J. Biochem., 85:157-162, 1979). Bovine pancreatic chymotrypsin (Boehringer Mannheim) was assayed using N-Suc-Ala-Ala-Pro-Phe-pNA (Sigma) in 100 mM Tris—HCl, pH 7.8, 10 mM CaCl₂ (DelMar, et al., Anal. Biochem., 99:316-320, 1979). Human neutrophil cathepsin G (Calbiochem-Novabiochem) was assayed using N-Suc-Ala-Ala-Pro-Phe-pNA (Sigma) in 625 mM Tris—HCl, pH 7.5, 2.5 mM MgCl₂, 0.125% Brij 35 (Groutas, et al., Arch. Biochem. Biophys., 294:144-146, 1992). Human plasma plasmin (Boehringer Mannheim) was assayed using Tosyl-Gly-Pro-Lys-pNA (Sigma) in 100 mM Tris—HCl, pH 7.4, 100 mM NaCl, 0.05% Triton X-100. Human plasma factor Xa (Calbiochem-Novabiochem) was assayed using N-Benzoyl-Ile-Glu-Gly-Arg-pNA (Pharmacia Hepar) in 50 mM Tris—HCl, pH 7.8, 200 mM NaCl, 0.05% BSA (Lottenberg, et al., Meth. Enzymol., 80:341-361, 1981). Human plasma thrombin (Boehringer Mannheim) was assayed using H-D-Phe-Pip-Arg-pNA (Pharmacia Hepar) in 50 mM Tris—HCl, pH 8.3, 100 mM NaCl, 1% BSA. Human plasma (Calbiochem-Novabiochem) and tissue kallikrein activities were assessed in 50 mM Tris-HCl, pH 7.8, 200 mM NaCl, 0.05% BSA using H-D-Prolyl-Phe-Arg-pNA (Pharmacia Hepar) and DL-Val-Leu-Arg-pNA (Sigma), respectively (Lottenberg, et al., Meth. Enzymol., 80:341-361,1981). The inhibition constants (Kᵢs) of human SLPI against each proteolytic enzyme were determined as previously described (Zitnik, et al., Biochem. Biophys. Res. Commun., 232:687-697, 1997.)

### Guinea Pig Airway Hyperresponsiveness

Male Hartley guinea pigs (Charles River Laboratories Inc., Wilmington, Mass.) were sensitized to ovalbumin by intraperitoneal injection with a 0.5 ml solution of 10 μg ovalbumin and 10 mg aluminum hydroxide in phosphate-buffered saline. Booster injections were administered on weeks three and five to ensure high titers of IgE and IgG1 (Andersson, P., Int. Arch. Allergy Appl. Immunol., 64:249-258, 1981). Seven to nine weeks after the initial injection, the animals were used to evaluate antigen-induced guinea pig airway responses.

In order to evaluate antigen-induced airway hyperresponsiveness in guinea pigs, a baseline histamine bronchoprovocation was initially conducted in unrestrained animals. Guinea pigs (450-600 g) were placed in a whole body plethysmograph (Buxco Electronics, Troy, N.Y.). The animals were exposed to five-second bursts of histamine aerosol generated by a DeVilbis ultrasonic nebulizer (Somerset, Pa.). The peak bronchoconstrictor response, expressed as Pause_{enhanced} (Chand, et al. Allergy, 48:230-235, 1993), was determined in response to rising histamine concentrations of 0, 25, 50, 100, and 200 mg/ml in phosphate-buffered saline (PBS) (GIBCO, Grand Island, N.Y.) administered at 10-minute intervals. Three days after the histamine baseline determination, the guinea pigs were again placed in the whole body plethysmograph and challenged with a three-second aerosolized burst of 0.1 mg/ml ovalbumin in phosphate-buffered saline. Six hours after antigen exposure, the development of hyperresponsiveness was evaluated by repeating the histamine bronchoprovocation.

SLPI was administered by intratracheal instillation in PBS (pH 7.2). After anesthetizing a guinea pig with inhaled methoxyflurane, an endotracheal tube (18 gauge Teflon® sheath) was visually passed into the trachea with the aid of a fiberoptic light source. SLPI (or PBS for control animals) was dosed through the tube, followed by a bolus of air to facilitate dispersion.

### Antigen-Induced Airway Responses in Sheep, Airway Mechanics

Adult ewes (median weight 30 kg) were instrumented as previously described (Abraham, et al., Eur. J Pharmacol. 217:119-126, 1992). Mean pulmonary flow resistance (R_{L}) was calculated from an analysis of 5 to 10 breaths by dividing the change in transpulmonary pressure by the change in flow at midtidal volume. Immediately after R_{L} determination, thoracic gas volume (V_{tg}) was measured in a constant volume body plethysmograph to calculate specific lung resistance (SR_{L}) by the equation SR_{L}=R_{L}×V_{tg}).

A Raindrop jet nebulizer (Puritan-Benett, Lenexa, Kans.), operated at a flow rate of 6 L/min, was used to generate droplets with a mass median aerodynamic diameter of 3.6±1.9 μm. Aerosol delivery was controlled using a dosimetry system which was activated for one second at the onset of the inspiratory cycle of a piston respirator (Harvard Apparatus Co., South Natick, Mass.). Aerosols were delivered at a tidal volume of 500 ml and a respiratory rate of 20 breaths per minute.

Ascaris-sensitive sheep which exhibited both early and late phase bronchoconstriction were challenged with Ascaris suum extract (82,000 protein nitrogen units/ml in phosphate-buffered saline) (Greer Diagnostics, Lenoir, N.C.) delivered as an aerosol at a rate of 20 breaths/minute for 20 minutes. Changes in SR_{L} were monitored for eight hours after antigen challenge.

### Airway Hyperresponsiveness

Baseline airway responsiveness was determined by measuring the SR_{L} immediately after saline inhalation and consecutive administration of 10 breaths of increasing concentrations of carbachol (0.25, 0.5, 1.0, 2.0, and 4.0% w/v). Airway responsiveness was estimated by determining the cumulative carbachol breath units required to increase SRL by 400% over the post-saline value (PC₄₀₀). One breath unit was defined as one breath of an aerosol containing 1% w/v carbachol. Antigen-induced airway hyperresponsiveness was determined by repeating the carbachol dose response 24 hours after antigen challenge.

### Tracheal Mucus Velocity

Restrained adult ewes were nasally intubated with an endotracheal tube (inside diameter 7.5 cm) (Mallinckrodt Medical Inc., St. Louis, Mo.) shortened by 6 cm. The cuff of the tube was place immediately below the vocal cords, as verified by fluoroscopy, to allow for maximal exposure of the tracheal surface. The inspired air was warmed and humidified using a Benett humidifier (Puritan-Benett, Lenexa, Kans.). The endotracheal tube cuff was inflated only during antigen and drug exposure to minimize physical impairment of tracheal mucus velocity.

Tracheal mucus velocity was quantified by fluoroscopy as previously described (O'Riordan, et al., Am. Rev. Respir. Crit. Care Med., 155:1522-1528, 1997). Five to ten radioopaque Teflon® particles (≈1 mm diameter, 0.6 mm thick, 1.5-2.0 mg) were insuflated into the trachea using a modified suction catheter connected to a source of compressed air at a flow rate of 3-5 L/min. Particle movement over a one minute period, detected by fluoroscopy, was recorded on videotape. The actual distance of particle movement was determined by a comparison with spaced radioopaque markers in an external collar.

### RESULTS

### Specificity Profile of SLPI

Characterization of the protease inhibitory activity of SLPI is summarized in Table 4. SLPI exhibits potent broad-spectrum inhibition of serine proteases implicated in asthma pathology, including cathepsin G, elastase, and tryptase. In contrast, factor Xa, kallikreins, thrombin, and plasmin were unaffected by SLPI at concentrations lower than 83 μM.

TABLE 4
Protease Inhibition Profile of SLPI

| Enzyme | Kᵢ (nM) |
|---|---|
| Chymotrypsin | 0.26 |
| Elastase | 0.34 |
| Tryptase | 0.58 |
| Cathepsin G | 11.0 |
| Trypsin | 23.6 |
| Kallikrein (tissue) | no inhibition at 83 μM |
| Thrombin | no inhibition at 83 μM |
| Factor Xa | no inhibition at 100 μM |
| Kallikrein (plasma) | no inhibition at 100 μM |
| Plasmin | no inhibition at 100 μM |

### Antigen-Stimulated Airway Hyperresponsiveness in Guinea Pigs

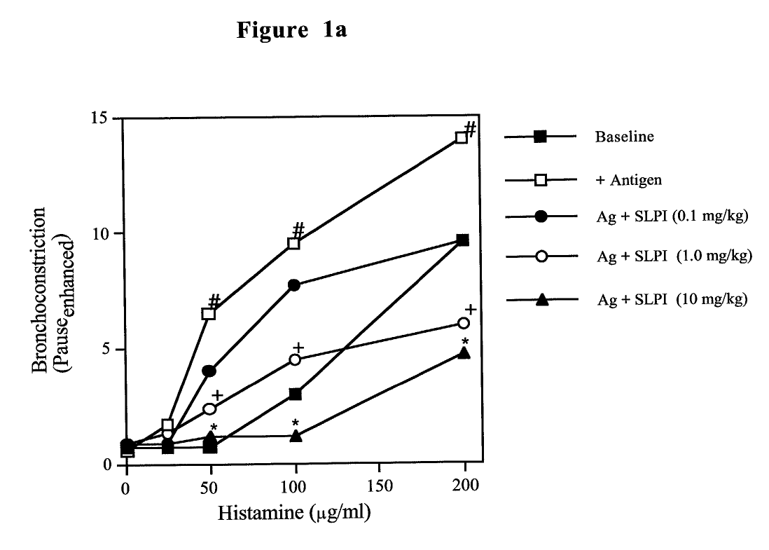

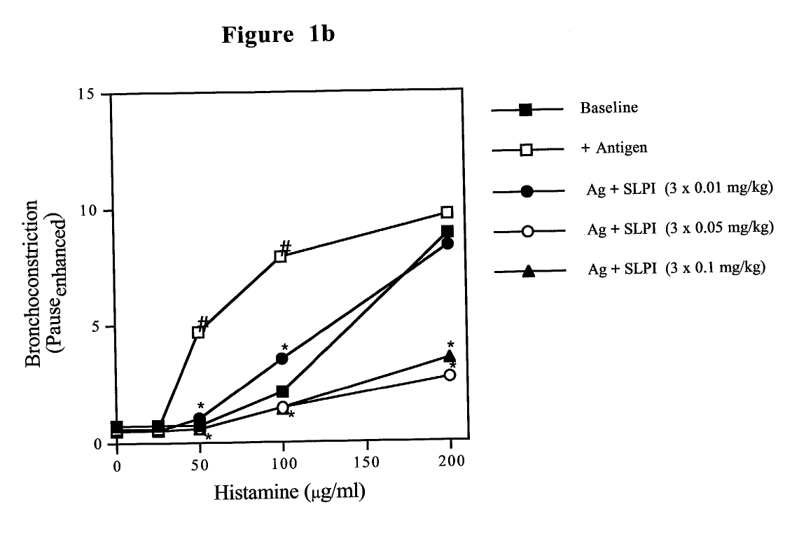

SLPI was evaluated for its effect on antigen-induced development of airway hyperresponsiveness in guinea pigs (FIG. 1). Six hours after antigen challenge, an increased pulmonary response to histamine bronchoprovocation was observed (n=4-10) (#p<0.05 vs. baseline histamine response). Intratracheal administration of SLPI one hour before antigen challenge provided a dose-dependent inhibitory effect against the development of hyperresponsiveness (FIG. 1a). SLPI inhibited the hyperreactivity to the 50 μg/ml dose of histamine with an ED₅₀ of 0.15 mg/kg, with a no effect dose of approximately 0.1 mg/kg. In contrast, predosing daily for two days with an additional dose one hour before antigen challenge reduced the ED₅₀ to <0.05 mg/kg (FIG. 1b).

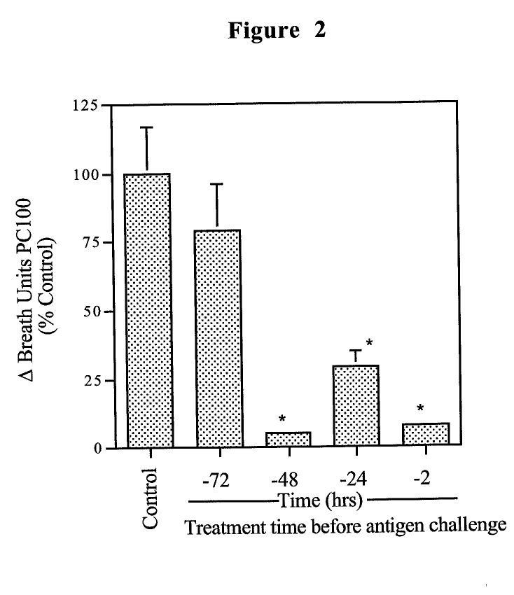

The duration of action of SLPI was also examined in the guinea pig. In this study, hyperresponsiveness was evaluated as the change in the histamine dose required to induce a 100% change in airway resistance (PC 100) 24 hours after antigen challenge (FIG. 2). Treatment with a single 5 mg intratracheal dose of SLPI 2, 24, or 48 hours before antigen challenge inhibited the development of airway hyperresponsiveness (n=4-10) (*p<0.05). However, no inhibitory effect was observed if SLPI was administered 72 hours before antigen challenge. These results demonstrate a prolonged pharmacodynamic effect of SLPI against antigen-induced airway hyperresponsiveness.

### Antigen-Stimulated Bronchial Responses in Sheep

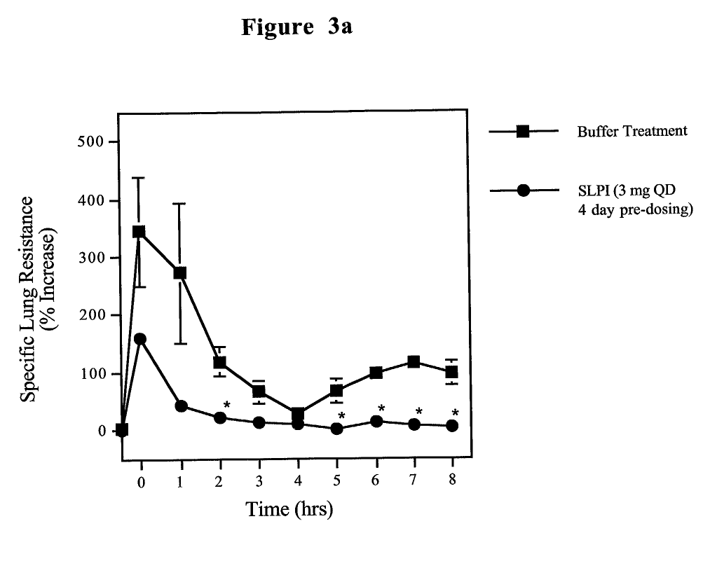

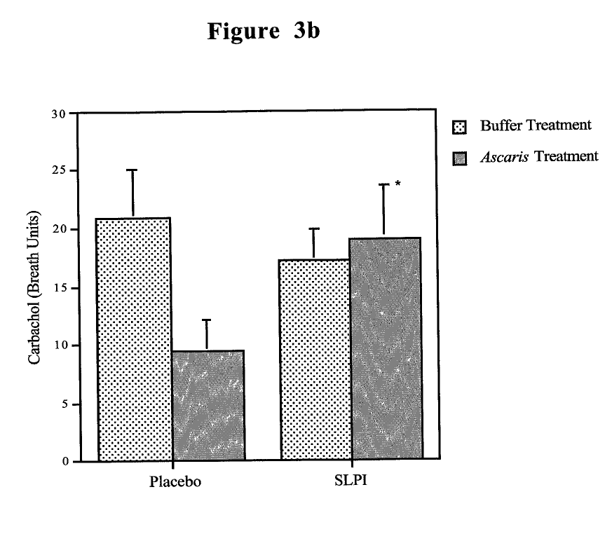

The effects of SLPI against antigen-induced early and late bronchoconstriction and the development of airway hyperresponsiveness were evaluated in a sheep bronchoprovocation model. SLPI (3 mg) preadministered daily for three days and 0.5 hour before antigen challenge (n=4) provided 48% and 100% inhibition of peak early-phase and late-phase bronchoconstriction, respectively (FIG. 3a) (*p<0.05 vs. antigen-stimulated bronchoconstriction). In addition, an 84% inhibition in the development of hyperresponsiveness was observed 24 hours after antigen challenge (FIG. 3b) (*p<0.05 vs. antigen-stimulated hyperresponsiveness). In comparison, a single dose of SLPI administered 0.5 hour before antigen challenge inhibited early and late phase responses with ED₅₀s of 76 and 48 mg, respectively, with a no effect dose of 10 mg (data not shown). The prophylactic regimen provided inhibitory activity equivalent to that achieved with a single 100 mg aerosol dose of SLPI administered 0.5 hour before antigen challenge. To further characterize the pharmacodynamics of SLPI activity in the sheep model, SLPI (3 mg) was administered every day for three days before antigen challenge, with the final dose 24 hours before antigen challenge (n=3). SLPI inhibited the peak late phase bronchoconstriction by 60% (*p<0.05 vs. antigen-stimulated bronchoconstriction), while no inhibition of the immediate response was observed.

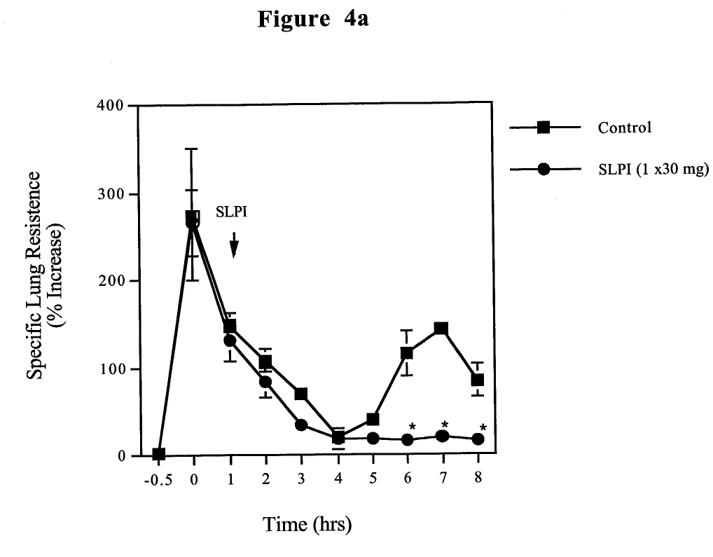

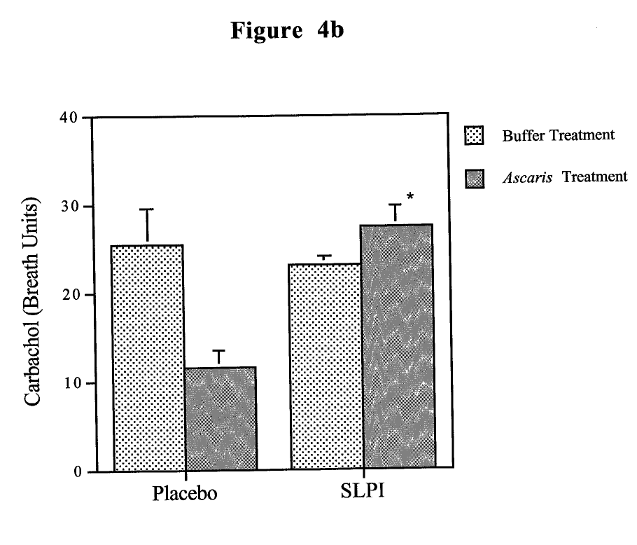

SLPI was also shown to be effective when administered after antigen challenge. As shown in FIG. 4, SLPI (30 mg) administered by aerosol one hour after antigen challenge, and the resultant peak early phase bronchoconstriction, is effective in inhibiting the subsequent late phase bronchoconstriction (n=5) (*p<0.05 vs. antigen-stimulated bronchoconstriction) (FIG. 4a) and development of airway hyperresponsiveness (n=5) (*p<0.05 vs. antigen-stimulated hyperresponsiveness) (FIG. 4b).

### Antigen-induced Effects on Tracheal Mucus Velocity in Sheep

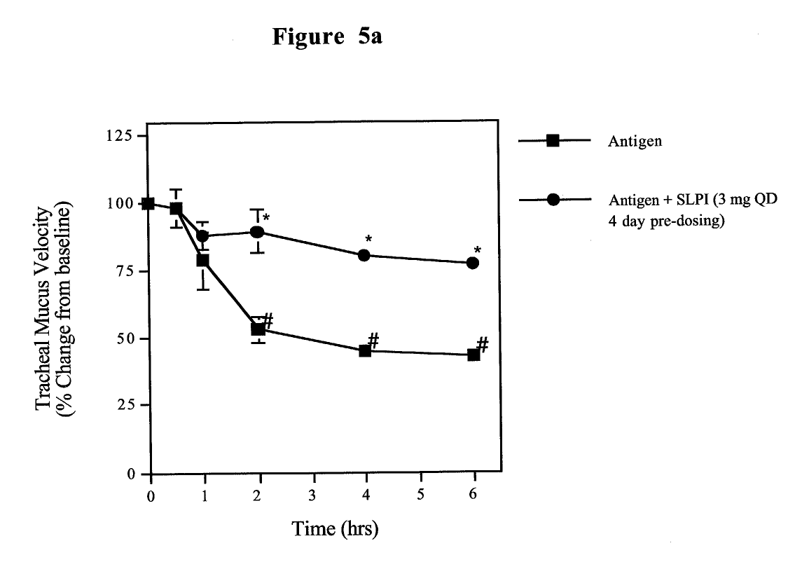

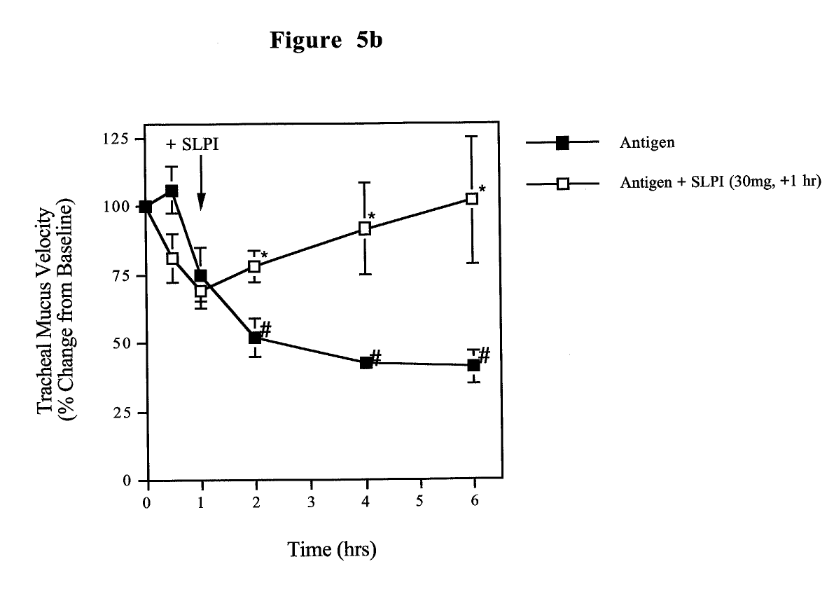

Antigen-induced effects on mucociliary function in sheep were assessed as a function of tracheal mucus velocity (FIG. 5). Beginning two hours after Ascaris challenge, significant reductions of tracheal mucus velocity were observed (n=3) (#p<0.05). After six hours, tracheal mucus velocity had decreased to 42% of the baseline response. SLPI (30 mg) alone had no effect on baseline velocity (data not shown). SLPI (3 mg) preadministered daily by aerosol for three days and 0.5 hour before antigen challenge (n=3) significantly inhibited the antigen-induced decrease in tracheal mucus velocity (*p<0.05) (FIG. 5a). This prophylactic regimen provided inhibitory activity equivalent to that achieved with a single 30 mg aerosol dose of SLPI administered 0.5 hour before antigen challenge. The single-administration no effect dose was 10 mg (data not shown). In addition, administration of 30 mg of SLPI one hour after antigen challenge reversed the decrease in tracheal mucus velocity (n=6) (*p<0.05) (FIG. 5b).

### DISCUSSION

SLPI represents a novel therapeutic approach to the treatment of asthma. SLPI is a broad spectrum serine protease inhibitor naturally produced in the human airway. These studies demonstrated that SLPI can provide effective therapy in preventing antigen-induced pathophysiologic airway responses, including early- and late-phase bronchoconstriction and development of airway hyperresponsiveness, and mucociliary dysfunction.

While asthma has not been associated with a deficiency of SLPI, mounting evidence demonstrates the development of a protease-antiprotease imbalance in the airways of asthmatic patients. Immediate mast cell responses as well as later leukocyte activation significantly increase the protease load in human airways following antigen exposure while the inflammatory milieu promotes SLPI inactivation. The resultant increase in proteolytic activity contributes to airway pathophysiology as well as the airway remodeling associated with asthma.

Broad spectrum serine protease inhibitory activity is crucial to the therapeutic utility of SLPI. SLPI provides potent broad spectrum inhibitory activity against mast cell and leukocyte serine proteases, including cathepsin G, elastase, and tryptase. In contrast, SLPI has no effect on factor Xa, thrombin, or plasmin, serine proteases whose chronic inhibition could have an adverse effect on coagulation and fibrinolysis.

Previous reports suggest that inhibition of a single serine protease is not sufficient to impact that pathophysiology and pathology associated with asthma. In sheep, α₁-protease inhibitor has been shown to prevent antigen-induced mucociliary dysfunction through inhibition of elastase (O'Riordan, et al., Am. Rev. Respir. Crit. Care Med., 155:1522-1528, 1997) and the development of airway hyperresponsiveness through inhibition of tissue kallikrein while having no effect on early or late phase bronchoconstriction (Forteza, et al., Am. J. Respir. Crit. Care Med. 1, 154:36-42, 1996). In contrast, tryptase inhibition prevents antigen-induced changes in pulmonary mechanics while having little impact on tracheal mucus velocity (unpublished data). In comparison, SLPI inhibits early and late phase bronchoconstriction, development of hyperresponsiveness and changes in mucociliary clearance following antigen challenge. Although SLPI fails to inhibit tissue kallikrein, inhibition of tryptase can prevent activation of prekallikrein as well as the direct release of bradykinin from kininogens.

The breadth of pharmacologic activity for SLPI is similar to that reported for corticosteroids. As shown with SLPI, steroid treatment inhibits changes in both pulmonary mechanics (Abraham, et al., Bull. Eur. Physiopathol. Respir.: Clin. Respir. Physiol., 22:387-392, 1986) and mucociliary function (O'Riordan, et al., Am. J. Respir. Crit. Care Med., 155:A878, 1997) in bronchoprovocation models. It is interesting to note that steroids have been shown to increase SLPI transcript levels in airway epithelial cell in vitro and airway levels of SLPI in vivo (Abbinante, et al., Am. J. Physiol. (Lung Cell. Mol. Physiol.), 12:L601-L606, 1995; Stockley, et al., Thorax, 41:442-447, 1986). While the relative contribution of SLPI elevation to the overall therapeutic activity of steroids is unknown, these observations indicate that SLPI may provide therapeutic activity similar to steroids without the associated systemic adverse effects.

Of particular interest is the pharmacodynamic activity of SLPI. A predosing regimen significantly reduces the amount of SLPI required to provide therapeutic activity. In the guinea pig model of airway hyperresponsiveness, SLPI had an ED₅₀ of 0.15 mg/kg when administered one hour before antigen challenge. In comparison, the ED₅₀ was reduced to <0.05 mg/kg when administered daily for two days before antigen challenge with an additional dose one hour before antigen challenge. Similar effects of pretreatment were observed in sheep, where a 3 mg dose of SLPI administered daily for three days before antigen challenge and 0.5 hour before antigen challenge (total dosage of 12 mg) had an inhibitory effect equivalent to single 100 mg and 30 mg doses administered 0.5 hour before antigen challenge in the bronchoconstriction and tracheal mucus velocity models, respectively. In addition, extended SLPI activity was observed in both guinea pigs and sheep models.

The improved efficacy of SLPI when administered in a predosing regimen may be accounted for, in part by its long half-life in the airway. The elimination half-life values of immunoreactive SLPI in the epithelial lining fluid in sheep and humans following aerosol administration are 12 and 6.5 hours, respectively (McElvaney, et al., Am. Rev. Respir. Dis., 148:1056-1060, 1993). Accumulation alone, however, cannot account for the efficacy of predosing, as the total doses given to guinea pigs or sheep approximates only the no effect doses for single administrations. An explanation may be that the predosing reduces the protease tone over several days to ameliorate the subsequent responses to antigen challenge, especially if proteases serve to prime the responses of mast cells and leukocytes. Additionally, the predosing period may provide sufficient time for tissue distribution to maximize its inhibitory activity (Dietze, et al., Biol. Chem. Hoppe-Seyler., 371 suppl.:75-79, 1990). As a result of intracellular compartmentalization of SLPI or distribution to the epithelial surface in the airways, half-life values determined from bronchial fluid may fail to fully quantify SLPI in the airway (Stolk et al., Thorax, 50:645-650, 1995).

Another important pharmacologic characteristic of SLPI is its ability to inhibit responses when administered after the initiation of airway responses. As shown in the sheep models, administration of 30 mg of SLPI one hour after antigen challenge and the resultant mast cell degranulation is capable of preventing the subsequent late phase bronchoconstriction, development of airway hyperresponsiveness as well as reversing the decrease of tracheal mucus velocity. These results demonstrate the potential utility of SLPI as a rescue therapy.

There is increased recognition of the need for agents which prevent airway remodeling to complement symptomatic relief in the treatment of asthma. The ability of SLPI to prevent mucociliary dysfunction represents an intervention against a critical pathologic change of the asthmatic airway. This observation is complemented by the ability of SLPI to inhibit elastase-induced bronchial secretory cell metaplasia (Lucey, et al., J. Lab. Clin. Med., 115:224-232, 1990) and mucus hypersecretion (King, et al., Am. J. Respir. Crit. Care. Med., 151:A529, 1995).

### Example 5

### Effect of SLPI Dry Powder Formulation on Antigen-Stimulated Bronchial Responses in Guinea Pigs

Male Hartley guinea pigs (Charles River Laboratories Inc., Wilmington, Mass.) were sensitized to ovalbumin by intraperitoneal injection with a 0.5 ml solution of 10 μg ovalbumin and 10 mg aluminum hydroxide in phosphate-buffered saline. Booster injections were administered on weeks three and five to ensure high titers of IgE and IgG1 (Andersson, P., Int. Arch. Allergy Appl. Immunol., 64:249-258, 1981). Seven to nine weeks after the initial injection, the animals were used to evaluate antigen-induced guinea pig airway responses.

In order to evaluate antigen-induced airway hyperresponsiveness in guinea pigs, a baseline histamine bronchoprovocation was initially conducted in unrestrained animals. Guinea pigs (450-600 g) were placed in a whole body plethysmograph (Buxco Electronics, Troy, N.Y.). The animals were exposed to five second bursts of histamine aerosol generated by a DeVilbis ultrasonic nebulizer (Somerset, Pa.). The peak bronchoconstrictor response, expressed as Pause_{enhanced} (Chand, et al., Allergy, 48:230-235, 1993) in response to rising histamine concentrations of 0, 25, 50, 100, and 200 mg/ml in phosphate-buffered saline (PBS) (GIBCO, Grand Island, N.Y.) administered at 10 minute intervals, was determined and the area under the curve (AUC) was calculated (n=16). Three days after the histamine baseline determination, the guinea pigs were again placed in the whole body plethysmograph and challenged with a three second aerosolized burst of 0.1 mg/ml ovalbumin in phosphate-buffered saline (n=16). Six hours after antigen exposure, the development of hyperresponsiveness was evaluated by repeating the histamine bronchoprovocation and calculating the AUC (#p<0.05 vs baseline response).

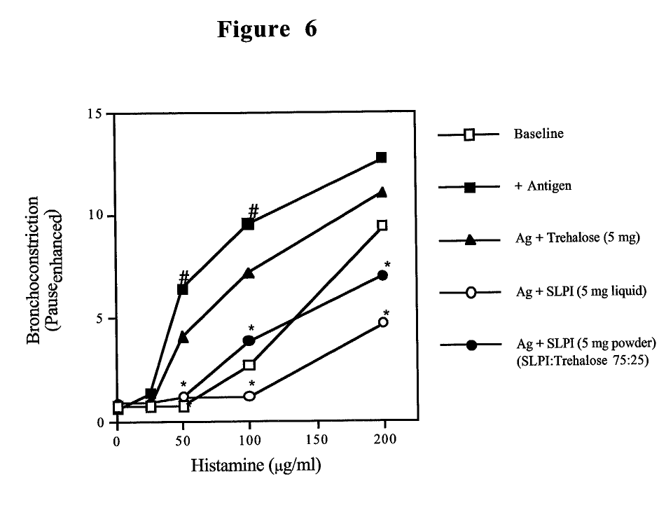

SLPI (5 mg) was administered as a liquid by intratracheal instillation (n=4) or as a SLPI:trehalose (75:25) powder (n=8) by intratracheal insufflation (FIG. 6). After anesthetizing a guinea pig with inhaled methoxyflurane, an endotracheal tube (18 gauge Teflon® sheath) was visually passed into the trachea with the aid of a fiberoptic light source. SLPI or trehalose (5 mg) (n=9) was dosed through the tube followed by a bolus of air to facilitate dispersion. The dry powder formulation of SLPI inhibited antigen-induced development of airway hyperresponsiveness equivalent to the effect of a similar amount of SLPI delivered intratracheally as a liquid (*P<0.05 vs antigen-stimulated response). In comparison, trehalose powder alone had no inhibitory effect on the antigen-stimulated response.

### Example 6

### Effect of SLPI Dry Powder Formulation on Antigen-Stimulated Bronchial Responses in Sheep

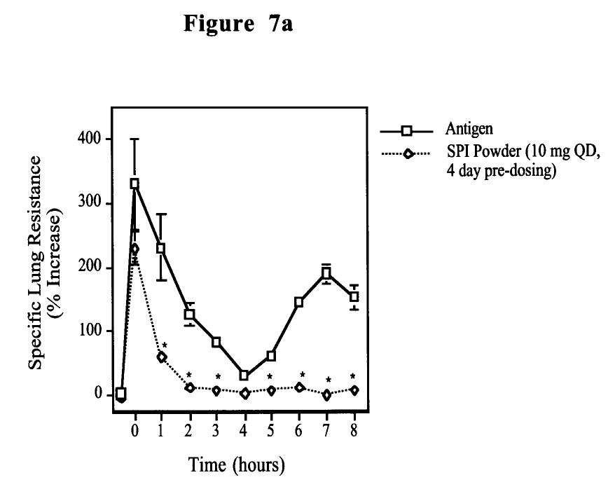

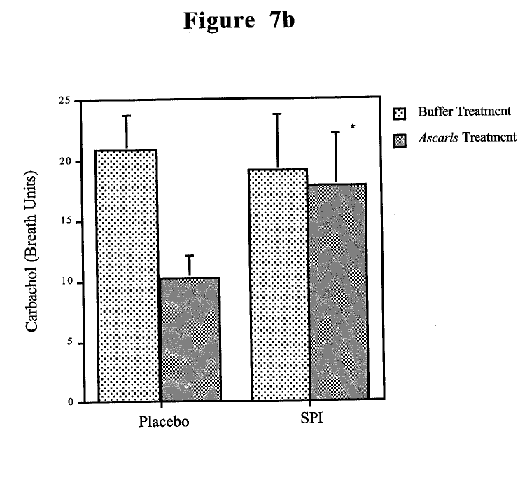

The effects of a dry powder formulation of SLPI against antigen-induced early and late bronchoconstriction and the development of airway hyperresponsiveness were evaluated in sheep (measurements were performed according to standard techniques as described in Abraham et al., J Clin Invest., 93:776-787, 1994). SLPI powder (10 mg, prepared as described above) was delivered to intubated sheep using a Rotohaler device. SLPI was administered daily for three days and 0.5 hour before antigen challenge (n=4). SLPI inhibited the early phase bronchoconstriction, measured as the area under the curve for the increase in specific lung resistance 0-4 hours after antigen challenge, by greater than 50% (FIG. 7a). SLPI also inhibited the late phase bronchoconstriction, with 100% inhibition of the peak response measured seven hours after antigen challenge (FIG. 7b) (*p<0.05 vs. antigen-stimulated bronchoconstriction). In addition, the SLPI powder formulation inhibited the development of airway hyper-responsiveness, measured 24 hours after antigen challenge, by 88% (*p<0.05 vs. antigen-stimulated hyperresponsiveness).

What is claimed is:

1. A pharmaceutical composition, comprising secretory leukocyte protease inhibitor (SLPI) protein and a pharmaceutically acceptable carrier, wherein said composition is a dry powder of less than about 10% by weight water, and wherein 50% to 95% by mass of said powder comprises particles or agglomerates of particles having a diameter within the range of from about 1.0 microns to about 8 microns with a mass median diameter ranging from about 3.0 microns to about 6 microns.

2. A pharmaceutical composition of claim 1, wherein said particles are at least 50% dispersible in a current of a gas.

3. A pharmaceutical composition of claim 1, wherein said particles or agglomerates of particles have a mass median diameter ranging from about 4.5 microns to about 5.5 microns.

4. A pharmaceutical composition of claim 1, comprising from about 50 to about 95 percent by weight SLPI protein.

5. A pharmaceutical composition of claim 1, comprising from about 5 to about 50 percent by weight pharmaceutically acceptable carrier.

6. A pharmaceutical composition of claim 1, wherein said pharmaceutically acceptable carrier is selected from the group consisting of carbohydrates, amino acids and polypeptides.

7. A pharmaceutical composition of claim 1, wherein said pharmaceutically acceptable carrier is selected from the group consisting of mannitol, sucrose, and trehalose.

8. A pharmaceutical composition of claim 1, further comprising a dispersing agent.

9. A pharmaceutical composition of claim 1, further comprising an absorption enhancer.

10. A pharmaceutical composition produced by the process comprising the steps of:
a) providing a mixture of secretory leukocyte protease inhibitor protein and optionally a pharmaceutically acceptable carrier in a solvent; and
b) spray drying said mixture to form a dry powder wherein said secretory leukocyte protease inhibitor comprises from about 50 to 100% by weight of the dry powder, wherein at least 50% to 95% by mass of the composition consists of particles or agglomerates of particles having a diameter within the range of from about 1.0 microns to about 8 microns with a mass median diameter ranging from about 3.0 microns to about 6 microns, and wherein the resulting composition is suitable for intratracheobronchial deposition.

11. A pharmaceutical composition of claim 10, wherein said particles are at least 50% dispersible in a current of a gas.

12. A pharmaceutical composition of claim 10, wherein said particles or agglomerates of particles have a mass median diameter ranging from about 4.5 microns to about 5.5 microns.

13. A pharmaceutical composition of claim 10, comprising from about 50 to about 95 percent by weight SLPI protein.

14. A pharmaceutical composition of claim 10, comprising from about 5 to about 50 percent by weight pharmaceutically acceptable carrier.

15. A pharmaceutical composition of claim 10, wherein said pharmaceutically acceptable carrier is selected from the group consisting of carbohydrates, amino acids and polypeptides.

16. A pharmaceutical composition of claim 10, wherein said pharmaceutically acceptable carrier is selected from the group consisting of mannitol, sucrose, and trehalose.

17. A pharmaceutical composition of claim 10, further comprising a dispersing agent.

18. A pharmaceutical composition of claim 10, further comprising an absorption enhancer.

19. A method of inhibiting a protease enzyme comprising the pulmonary administration of a pharmaceutical composition of claim 1 or 10.

20. A method of claim 19, wherein said composition is administered daily.

21. A method according to claim 19, comprising directing said pharmaceutical composition into the oral cavity of a patient while the patient is inhaling.

22. A method of inhibiting pulmonary mucous production/secretion comprising the pulmonary administration of a pharmaceutical composition of claim 1 or 10.

23. A method of increasing mucous velocity in the airways comprising the pulmonary administration of a pharmaceutical composition of claim 1 or 10.

24. A method of decreasing airway hyperresponsiveness to antigen/stimulus comprising the pulmonary administration of a pharmaceutical composition of claim 1 or 10.

25. A method of inhibiting pathological changes to airway cells/tissue comprising the pulmonary administration of a pharmaceutical composition of claim 1 or 10.
